(19) United States
(12) Patent Application Publication
(10) Pub. No.: US 2007/0041944 A1
(43) Pub. Date: Feb. 22, 2007

(54) Treating tumors by ENH dislocation of ID proteins

(75) Inventors: Antonio Iavarone, New York, NY (US); Anna Lasorella, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/416,277

(22) Filed: May 1, 2006

Related U.S. Application Data
(60) Provisional application No. 60/677,930, filed on May 5, 2005.

(51) Int. Cl. A61K 48/00; C12Q 1/68; G01N 33/574; C12N 15/86; A61K 9/127

(52) U.S. Cl. 424/93.2; 435/7.23; 435/456; 977/802; 435/6; 424/450

(57) ABSTRACT

The invention provides methods for the therapy of cancer that are directed towards preventing or inhibiting Id function. The present methods are based on contacting neoplastic or tumor cells with agents that can translocate an Id protein to the cytoplasm and/or sequester an Id protein in the cytoplasm, thereby preventing Id-mediated promotion of cell cycle activation and/or Id-mediated repression of cell differentiation. The invention also provides methods for screening agents that can be useful in the methods of treatment. The agents used in the methods can be, for example, proteins that possess: (1) at least one LIM domain, (2) at least one PDZ domain, or (3) at least one LIM domain and at least one PDZ domain. One particular agent that is used in the methods is the ENH (Enigma Homologue) protein, or peptides or peptidomimetics thereof. Because binding to an Id protein can be mediated by the LIM domain of the ENH protein, and given the high degree of structural conservation existing among the family of proteins to which ENH belongs (the PDZ-LIM protein family), the invention provides at least the members, mutants and variants of the PDZ-LIM protein family as agents that are capable of sequestering an Id protein in the cytoplasm.

This applications claims priority to U.S. Ser. No. 60/677,930, filed May 5, 2005, which is hereby incorporated by reference in its entirety.

The invention disclosed herein was made in part with U.S. Government support from the National Institutes of Health/National Cancer Institute grant numbers R01-CA85628 and R01-CA101644. Accordingly, the U.S. Government may have certain rights in this invention.

This disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety.

### BACKGROUND OF THE INVENTION

Id (inhibitor of DNA binding or inhibitor of differentiation) proteins belong to the helix-loop-helix (HLH) protein superfamily that is composed of seven currently known subclasses. The dimerization of basic HLH proteins is necessary for their binding to DNA at the canonical E-box (CANNTG) or N-box (CACNAG) recognition sequences. Id proteins lack the basic domain necessary for DNA binding, and act primarily as dominant-negative regulators of bHLH transcription factors by sequestering and/or preventing DNA binding of ubiquitously expressed (e.g., E12, E47, E2-2) or cell-type-restricted (e.g., Tal-1, MyoD) factors. Four members of the Id protein family (Id1 to Id4) have been identified in mammals. All Id proteins share a highly homologous HLH region, but have divergent sequences elsewhere.

Id proteins generally act as negative regulators of differentiation, and depending on the specific cell lineage and developmental stage of the cell, Id proteins can act as positive regulators. Because bHLH proteins are mainly involved in the regulation of the expression of tissue specific and cell cycle related genes, Id-mediated sequestration or repression of bHLH proteins serves to block differentiation and to promote cell cycle activation. Accordingly, Id proteins have been shown to have biological roles as coordinators of different cellular processes, such as cell-fate determination, proliferation, cell-cycle regulation, angiogenesis, and cell migration. The present invention provides new methods for inhibiting proliferation of a neoplastic cell and for inhibiting angiogenesis in tumor tissue

### SUMMARY OF THE INVENTION

The invention provides methods for treating cancer by contacting tumor cells with an agent that is capable of sequestering an Id protein in the cytoplasm of a cell and/or of translocating an Id protein from the nucleus to the cytoplasm of a cell. In various aspects, an agent that is capable of translocating and/or sequestering Id proteins to/in the cytoplasm is a protein that comprises either: (1) at least one LIM domain, (2) at least one PDZ domain, or (3) at least one LIM domain and at least one PDZ domain. In one aspect, the agent comprises or consists essentially of the ENH (Enigma Homologue) protein, or peptides or peptidomimetics thereof. Because binding to an Id protein can be mediated by the LIM domain of the ENH protein, and given the high degree of structural conservation existing among the family of proteins to which ENH belongs (the PDZ-LIM protein family), the invention provides at least the members, mutants and variants of the PDZ-LIM protein family can be agents that are capable of translocating and/or sequestering Id proteins in the cytoplasm.

In one aspect, the invention provides the actin cytoskeleton-associated PDZ-LIM protein ENH as an Id-associated protein. In one aspect, ENH, whose expression increases during neural differentiation, sequesters an Id protein in the cytoplasm and prevents cell-cycle progression and inhibition of bHLH transcription driven by the Id protein. In another aspect, silencing of ENH, for example, by RNA interference abolishes the relocation of an Id protein to the cytoplasm. In certain aspects, the invention relates to an antiproliferative and differentiation signaling pathway in the nervous system that converges upon the regulation of ENH, where this pathway prevents nuclear retention of Id2 and relieves the inhibitory constraints imposed by Id2 on nuclear transcription factors.

In one aspect, the invention provides a method for inhibiting proliferation of a cell or for promoting differentiation of the cell, the method comprising expressing in the cell a protein comprising at least one LIM domain(s), wherein the protein sequesters an Id protein in the cytoplasm of the cell, thereby inhibiting proliferation.

In one aspect, the invention provides a method for inhibiting proliferation of a neoplastic cell or for promoting differentiation of the neoplastic cell, the method comprising expressing in the neoplastic cell a protein comprising at least one LIM domain(s), wherein the protein sequesters an Id protein in the cytoplasm of the cell, thereby inhibiting proliferation.

In another aspect, the invention provides a method for inhibiting angiogenesis in a tumor, the method comprising expressing in a cell of the tumor a protein comprising at least one LIM domain, wherein the protein sequesters an Id protein in the cytoplasm of the cell, thereby inhibiting angiogenesis.

In another aspect, the invention provides a method for treating cancer in a subject, the method comprising administering to the subject an effective amount of a protein comprising at least one LIM domain, or an effective amount of a nucleic acid comprising a coding sequence for the protein, wherein the protein sequesters an Id protein in the cytoplasm of a cancer cell in the subject; thereby treating cancer in the subject. The protein can be administered to the subject, for example, in a liposome, virosome, or in a medical device. The nucleic acid can comprise, for example, an expression vector. The expression vector can comprise a viral vector, for example, an adenovirus (ADV) vector, an alphavirus vector or an adenovirus-associated virus (AAV) vector. Further, the nucleic acid can comprise, for example, a recombinant genome of a virus particle, wherein the virus particle is administered to the subject.

In certain aspects, the invention provides a protein comprising at least one LIM domain, wherein the LIM domain(s) can bind to an Id protein. In another aspect, where a protein can bind to an Id protein, the invention provides that the resultant protein-Id complex can be greater than about 40 kilodaltons, such that this complex (and therefore the Id protein) is prevented from migrating or being transported into the nucleus of the cell, thereby sequestering the Id protein in the cytoplasm. In another aspect, the invention provides a protein comprising at least one LIM domain and/or at least one PDZ domain, wherein the protein translocates an Id protein from the nucleus to the cytoplasm of the cell, thereby sequestering the Id protein in the cytoplasm.

In one aspect, the invention provides a method for treating cancer in a subject, the method comprising administering to the subject an effective amount of a chemical compound, wherein the chemical compound sequesters an Id protein in the cytoplasm of a cancer cell and/or translocates an Id protein from the nucleus to the cytoplasm of the cancer cell in the subject, thereby treating cancer in the subject.

In all aspects of the invention, where the methods relate to cells, tumor cells, cancer cells, neoplastic cells, or tumor or cancer tissue, these cells or tissue can be from or comprise a small cell carcinoma, a melanoma, a hepatocellular carcionoma, a colorectal adenocarcinoma, a pancreatic cancer, a medullary thyroid cancer, a papillary thyroid cancer, an astrocytic tumor, a neuroblastoma, Ewing's sarcoma, an ovarian tumor, a cervical cancer, an endometrial carcinoma, a breast cancer, a prostate cancer, a nervous system tumor, Wilms' tumor, a retinoblastoma, or a malignant seminoma. Further, where the methods are treatment methods, the subject treated can suffer from one of the above-listed tumors/cancers.

In all aspects of the invention, for a protein comprising at least one LIM domain, the protein can further comprise at least one PDZ domain. In certain aspects, the LIM domans(s) can bind to an Id protein, and the PDZ domain(s) can bind to a cytoskeletal protein. In certain aspects, a protein can sequester an Id protein in the cytoplasm by binding to the protein through a LIM domain(s) and simultaneously binding to a cytoskeletal protein through a PDZ domain(s). Proteins that comprise at least one PDZ domain and/or at least one LIM domain can comprise a PDZ-LIM protein, including a mutant or variant thereof. PDZ-LIM proteins include, but are not limited to, ALP, RIL, CLP-36/hClim1/Elfin, Mystique, Enigma/LMP-1, ENH, ZASP/Cypher1, LIMK-1, LIMK-2, LMO7, and mutants or variants (including isoforms) thereof. Isoforms of human ENH include isoform a (SEQ ID NO:4), isoform b (SEQ ID NO:6), isoform c (SEQ ID NO:8), isoform d (SEQ ID NO:10), and isoform e (SEQ ID NO:12).

Further, the invention provides recombinant proteins that are designed or derived from PDZ-LIM proteins, or from other LIM-containing or PDZ-containing proteins that are not members of the PDZ-LIM protein family.

A PDZ domain of the invention can comprise, for example, a sequence that is at least 75%, 80%, 85%, 90%, 95%, or 99% identical to: residues 10-82 of SEQ ID NO:4; residues 10-82 of SEQ ID NO:6; residues 10-82 of SEQ ID NO:6; residues 10-82 of SEQ ID NO:8; residues 10-82 of SEQ ID NO:10; or residues 10-82 of SEQ ID NO:12.

A LIM domain of the invention can comprise, for example, a sequence that is at least 75%, 80%, 85%, 90%, 95%, or 99% identical to: residues 420-470 of SEQ ID NO:4; residues 479-529 of SEQ ID NO:4; residues 537-590 of SEQ ID NO:4; residues 311-361 of SEQ ID NO:6; residues 370-420 of SEQ ID NO:6; or residues 428-481 of SEQ ID NO:6.

In all aspects of the invention, with respect to a protein comprising at least one LIM domain, wherein the LIM domain binds to an Id protein, the Id protein can be Id1, Id2, Id3 or Id4. In one aspect, the Id protein is Id2.

In one aspect, the invention provides a method for grading a tumor, the method comprising determining whether a PDZ-LIM protein is expressed in a tumor cell, wherein if the PDZ-LIM protein is expressed then the tumor is graded higher or as a more aggressive tumor than if the PDZ-LIM is not expressed. In one aspect, the PDZ-LIM protein is ENH.

In one aspect, the invention provides a method for identifying an agent that can sequester an Id protein in the cytoplasm of a cell and/or translocate an Id protein to the cytoplasm of the cell, the method comprising: (a) incubating the agent with the cell, wherein an Id protein is present in the nucleus of the cell prior to incubation; and (b) determining whether the Id protein of the cell is sequestered in and/or translocated to the cytoplasm of the cell, wherein if the Id protein is determined to be sequestered in and/or translocated to the cytoplasm, then the agent is identified as an agent that can sequester an Id in the cytoplasm and/or translocate an Id protein from the nucleus to the cytoplasm of a cell. In one aspect, the cell comprises a cancer cell. The cancer cell can comprise, for example, a cell from a small cell carcinoma, a melanoma, a hepatocellular carcionoma, a colorectal adenocarcinoma, a pancreatic cancer, a medullary thyroid cancer, a papillary thyroid cancer, an astrocytic tumor, a neuroblastoma, Ewing's sarcoma, an ovarian tumor, a cervical cancer, an endometrial carcinoma, a breast cancer, a prostate cancer, a nervous system tumor, Wilms' tumor, a retinoblastoma, or a malignant seminoma. In one aspect, the cell comprises a neuroblastoma cell. In one aspect, the Id protein is Id2. In one aspect, the determining step comprises immunofluorescence. In one aspect, the incubating step comprises incubating the agent with the cell from between about 30 minutes to about 72 hours.

In one aspect, the invention provides a medical device comprising a protein comprising a LIM domain, or a chemical compound, wherein the protein or the chemical compound sequesters an Id protein in the cytoplasm of a cell and/or translocates an Id protein to the cytoplasm of a cell. The medical device can comprise, for example, a stent, a transdermal patch, or an implantable biodegradable gel. In one aspect, the protein comprises ENH, or a mutant or a variant thereof. In one aspect, the Id protein is Id2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents members of the ALP and Enigma protein subfamilies of the PDZ-LIM protein family. The depicted proteins are human proteins. The abbreviation “aa” stands for amino acids.

FIG. 2 shows that ENH binds Id2 in vitro and in vivo through LIM domains (see Example 1). FIG. 2A is a schematic representation of full-length ENH and deletion mutants tested in binding experiments. The C-terminal region of ENH contained in the two clones identified from the two-hybrid screening is shown in the region demarcated by amino acid residues 461-596 of ENH. FIG. 2B shows the results of binding experiments where in vitro translated ³⁵S labeled full-length ENH and LIM domain deletion mutants were mixed with fusion proteins GST, GST-Id2, GST-Id2 lacking the HLH domain (GST-δHLHId2), GST-Id1 and GST-Id3. Bound proteins were analyzed by autoradiography. In the right panel in FIG. 2B, the lane “Input” shows in vitro-translated ³⁵S-labeled full-length ENH. For FIG. 2C and FIG. 2D, Cos-1 cells were transfected with the indicated expression plasmids. Lysates were analyzed directly (input) or immunoprecipated with antibodies against Id2 (FIG. 2C) or Flag (FIG. 2D). Immunoprecipitated proteins were analyzed by Western blot for Flag (FIG. 2C) or 1d2 (FIG. 2D). “NRIg” stands for Normal Rabbit Immunoglobulins.

FIG. 3 (see Example 1) shows that ENH is expressed in muscle and neural tissues and is up-regulated in neuroblastoma cells treated with retinoic acid (RA). Thus, differentiation of neuroblastoma cells with retinoic acid (RA) induces expression of ENH without changing the expression levels of Id2. FIG. 3A: Western blot analysis shows specificity of the ENH antibody for endogenous ENH in RA-treated SK-N-SH cells and ectopically expressed Flag-ENH. The ENH band is lost after treatment of the cells with siRNA oligonucleotides to ENH (siENH). SiCtr is a smart-pool siRNA mixture to luciferase (Dharmacon, Inc., Lafayette, Colo.). FIG. 3B: ENH immunohistochemistry from E15.5 mouse embryo shows expression in neural and muscle tissues. Magnification: X20 (Upper panels) and X100 (Lower panels). FIG. 3C presents a Northern Blot analysis of ENH and Id2 in SH-F and SH-N neuroblastoma cells treated with RA for the indicated times. 28S rRNA is shown as a loading control. FIG. 3D shows lysates from parallel cultures that were analyzed by Western Blot using ENH and Id2 antibodies. The asterisk indicates a non-specific band.

FIG. 4 shows that ENH relocates or translocates or sequesters Id2 to or in the cytoplasm. FIG. 4A shows that RA treatment induces expression of ENH and cytoplasmic relocation of Flag-Id2. SK-N-SH cells stably expressing Flag-Id2 were treated with RA or with vehicle control for 48 hours. Cells were double immunostained for Flag (green) and ENH (red). Nuclei were counterstained with DAPI. Arrows indicate cells showing coexpression of cytoplasmic Flag-Id2 and ENH. Arrowheads indicate cells with nuclear Flag-Id2 that lack ENH. In FIG. 4B, SK-N-SH cells were transiently transfected with Flag-ENH and immunostained for endogenous Id2 (red) and Flag (green). Nuclei were counterstained with DAPI. FIG. 4C shows a quantitative analysis of SK-N-SH cells displaying cytoplamsic Flag-Id2 from the experiments shown in FIG. 4A (at least 300 cells were scored for each sample).

FIG. 5 shows that ENH knockdown prevents translocation of Id2 to the cytoplasm in neuroblastoma cells treated with RA. FIG. 5A: Control (scrambled) or ENH-specific siRNA oligonucleotides were introduced in SK-N-SH expressing Flag-Id2 before treatment with RA or vehicle control for 72 h. Cells were immunostained for Flag-Id2 (red) and counterstained with DAPI. Arrowheads indicate cells displaying full relocation of Flag-Id2 to the cytoplasm after treatment with RA. FIG. 5B: Quantitative analysis of cells displaying predominant nuclear Flag-Id2 (at least 500 cells were scored for each sample).

FIG. 6 shows that ENH inhibits proliferation (and can overcome Id2-mediated entry into S-phase) and inhibits Id2-mediated functions. FIG. 6A: IMR32 neuroblastoma cells were transfected with a multimerized E-box-luciferase plasmid plus expression plasmids for the indicated proteins. Contransfection of increasing concentrations of ENH (0.375, 0.5, and 0.625 μg) relieved transcriptional inhibition by Id2. Results of luciferase activity are expressed as means of quadruplicate assays normalized for transfection efficiency by using β-galactosidase (error bars indicate standard deviations). FIG. 6B: SK-N-SH, IMR-32 (neuroblastoma), and SF188 (glioma) were transfected with ENH or the empty vector, and colonies were scored after selection in G418. The total number of colonies recovered from the empty vector control transfection of each cell line were as follows: SF188=168 colonies; IMR32=223 colonies; SK-N-SH=121 colonies. FIG. 6C: SK-N-SH cells were transfected with the indicated plasmid combinations. A plasmid encoding GFP was included to identify transfected cells. Cultures were labeled with BrdU for 6 h and 14 h and immunostained for BrdU by using a Cy3-conjugated secondary antibody. Cells were assessed for GFP and BrdU, and the percentage of transfected cells positive for BrdU was scored.

FIG. 7 shows that cytoplasmic translocation of Flag-Id2 in Nestin-Flag-Id2 transgenic mouse brain is associated with expression of ENH in differentiating cells. FIG. 7A: Western blot of E15.5 (embryo at day 15.5) brains from two transgenic lines (lanes 1-5, line 1; lanes 6-12, line 2) for Flag-Id2 shows expression of Flag-Id2 in hemizygous transgenic embryos (lanes 2 and 5-10) but not wild-type embryos (lanes 1, 3, 4, 11, and 12). Lane 13 is the positive control of SK-N-SH expressing Flag-Id2. α-Tubulin is shown as a control for loading. FIG. 7B: Lysates from whole brain of Nestin-Flag-Id2 transgenic (T) and control (NT) pups were immunoprecipitated with anti-Flag M2 antibody and analyzed for ENH and Flag-Id2 by Western blot. FIG. 7C: Adjacent sections from the brain of E15.5. Nestin-Flag-Id2 embryos were immunostained for Flag and ENH. Magnification: X20 (left panels) and X100 (center and right panels). Nuclear Flag-Id2 is detected in the VZ, which expresses barely detectable ENH. Strong expression of ENH in the MZ is associated with cytoplasmic relocation of Flag-Id2.

FIG. 8 shows that Id2 and ENH localize to the cytoplasm of differentiated tumor cells in glianeuroblastoma (GNB) cells. Id2 and ENH antibodies were used to immunostain primary tumors (brown precipitates). Magnification is X20 (upper panels) and X100 (lower panels).

FIG. 9A-9B presents the nucleotide sequence (SEQ ID NO:3) that codes for the amino acid sequence of human ENH isoform a (SEQ ID NO:4).

FIG. 10 presents the amino acid sequence (SEQ ID NO:4) of human ENH isoform a. This protein possesses a PDZ domain at about residues 10-82, and LIM domains (cysteine-rich double zinc fingers involved in protein-protein interactions) at residues 420-470, 479-529, and 537-590.

FIG. 11 presents the nucleotide sequence (SEQ ID NO:5) that codes for the amino acid sequence of human ENH isoform b (SEQ ID NO:6). This variant uses an alternate in-frame splice site compared to variant 1 (RNA for ENH isoform a) that result in an isoform (isoform b) that has the same N— and C-termini but is shorter compared to isoform a.

FIG. 12 presents the amino acid sequence (SEQ ID NO:6) of human ENH isoform b. This protein possesses a PDZ domain at about residues 10-82, and LIM domains at residues 311-361, 370-420, and 428-481.

FIG. 13 presents the nucleotide sequence (SEQ ID NO:7) that codes for the amino acid sequence of human ENH isoform c (SEQ ID NO:8). This variant lacks a coding exon compared to variant 1, that causes a frameshift. The resulting predicted isoform c is shorter and has a distinct C-terminus compared to isoform a.

FIG. 14 presents the amino acid sequence (SEQ ID NO:8) of human ENH isoform c. This protein possesses a PDZ domain at about residues 10-82.

FIG. 15 presents the nucleotide sequence (SEQ ID NO:9) that codes for the amino acid sequence of human ENH isoform d (SEQ ID NO:10). This variant differs in the 3′ UTR (not shown) and coding region compared to variant 1. The resulting isoform d is shorter, has internal differences, and has a distinct C-terminus compared to isoform a.

FIG. 16 presents the amino acid sequence (SEQ ID NO:10) of human ENH isoform d. This protein possesses a PDZ domain at about residues 10-82.

FIG. 17 presents the nucleotide sequence (SEQ ID NO:11) that codes for the amino acid sequence of human ENH isoform e (SEQ ID NO:12). This variant differs in the 3′ UTR (not shown) and coding region compared to variant 1. The resulting isoform e is shorter, lacks an internal segment, and has a distinct C-terminus compared to isoform a.

FIG. 18 presents the amino acid sequence (SEQ ID NO:12) of human ENH isoform e. This protein possesses a PDZ domain at about residues 10-82.

### DETAILED DESCRIPTION OF THE INVENTION

The invention provides methods for the therapy of cancer that are directed towards preventing or inhibiting Id function. The present methods are based on contacting neoplastic or tumor cells with agents that can translocate an Id protein to the cytoplasm and/or sequester an Id protein in the cytoplasm, thereby preventing Id-mediated promotion of cell cycle activation and/or Id-mediated repression of cell differentiation. The invention also provides methods for screening agents that can be useful in the methods of treatment.

### Terms

As used herein, “sequestering” means the end result of an action where an entity, such as an Id protein, is kept in a place, or is prevented from leaving a place, or is brought into a place, or is prevented from going into a second place. Therefore, “sequestering” an Id protein in the cytoplasm of a cell can mean, for example, retaining an Id protein in the cytoplasm, anchoring an Id protein in the cytoplasm, tethering an Id protein in the cytoplasm, relocating an Id protein from the nucleus to the cytoplasm, and translocating an Id protein from the nucleus to the cytoplasm of a cell. Thus, methods of the invention can be described as, translocating an Id protein to, and/or sequestering an Id protein in, the cytoplasm. Further, agents that can sequester an Id protein are also referred to as “sequestering and/or translocating” agents. Without being bound by theory, sequestering an Id protein can be an end result or effect that an agent has upon a cell, where the agent can: (1) bind to an Id protein in the cytoplasm and prevent the Id protein from diffusing into the nucleus because the size of the Id protein-agent complex is too large (about greater than 40 kilodaltons) to freely diffuse through nuclear pore complexes, (2) bind to an Id protein in the cytoplasm and prevent the interaction of Id with a protein that can transport Id to the nucleus, (3) bind to an Id protein in the cytoplasm, where the agent also binds to a structure or protein in the cytoplasm, such that the agent tethers the Id protein to the cytoplasmic structure or protein, and/or (4) bind to an Id protein in the nucleus and transport the Id protein out of the nucleus into the cytoplasm, i.e., translocate the Id protein out of the nucleus into the cytoplasm. Further, with respect to (4), where an agent translocates an Id protein to the cytoplasm, it is not necessarily the case that the agent thereafter retains the Id protein in the cytoplasm. Rather, it can be the case that the agent modifies the kinetics of Id nuclear import and export, such that the rate of export out of the nucleus is higher than the rate of import into the nucleus.

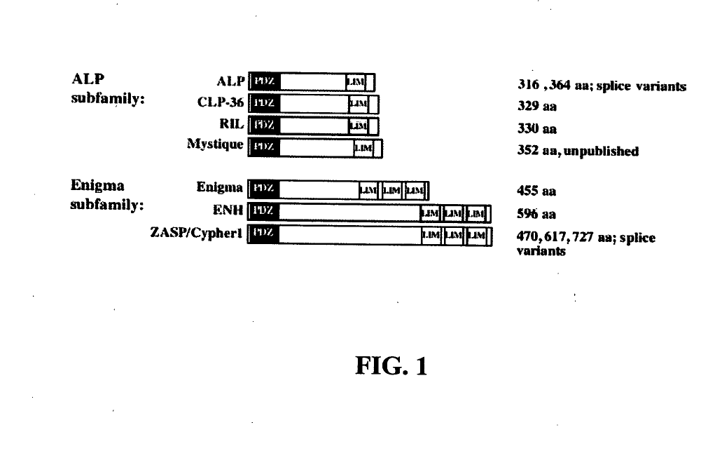

As used herein, a “LIM-PDZ protein family” member, or “LIM-PDZ” protein, refers to a naturally occurring group of proteins (and homologues, mutants, variants thereof) that share a high degree of amino acid similarity in their PDZ and LIM protein domains (up to 70% sequence similarity). The family now contains seven proteins (see FIG. 1), each of which contains one N-terminal PDZ domain followed either by one C-terminal LIM domain (ALP subfamily; ALP, RIL, CLP-36/hClim1/Elfin, Mystique) or three C-terminal LIM domains (Enigma subfamily; Enigma/LMP-1, ENH, ZASP/Cypher1) (Xia et al., J. Cell Biol., 271: 15934-15941, 1997).

As used herein, a “LIM/PDZ protein” is a protein that comprises either (a) at least one LIM domain, (b) at least one PDZ domain, or (c) at least one LIM domain and at least one PDZ domain. This definition of a “LIM/PDZ protein” therefore encompasses (a) all members of the “LIM-PDZ protein family”, as well as (b) mutants and variants thereof, where mutants can include recombinant proteins that comprise combinations of different LIM and/or PDZ domains from different LIM-PDZ family members, and (c) recombinant proteins that comprise LIM and/or PDZ domains from proteins that are LIM-PDZ family members or from proteins that are not LIM-PDZ family members; further, the recombinant proteins can include LIM and/or PDZ domains that have been derived from mutation methods such as directed evolution methods. The specific nomenclature of “LIM/PDZ” is used in order to differentiate the term from the “LIM-PDZ” protein family, because LIM/PDZ encompasses a greater scope of members.

As used herein, an “agent” refers to an entity that can sequester an Id protein(s) in the cytoplasm of a cell. Agents can be, for example, chemical compounds, nucleic acids, proteins, and peptides. Where the invention refers to agents that are not the active ingredient in compositions used in the methods of treatments mentioned herein, then the agent is referred to as an “inactive-agent” or “excipient.”

As used herein, a “peptidomimetic” is a chemical compound that mimics the ability of a peptide to recognize certain physiological molecules, such as proteins and DNA. In one embodiment, a peptidomimetic is a chemical compound that reproduces the structure of a LIM domain. In one embodiment, a peptidomimetic is a chemical compound that reproduces the structure of a LIM domain of a PDZ-LIM protein.

### Id Proteins

The invention provides methods for cancer therapy by targeting Id proteins and manipulating their subcellular localization. Specifically, the invention provides methods for sequestering Id proteins in the cytoplasm and/or translocating Id proteins to the cytoplasm. Therefore, all four mammalian member of the Id protein family, Id1, Id2, Id3 and Id4, are targets of the present methods.

All Id proteins share a highly homologous HLH region, whereas the rest of the sequences diverge among the members. However, the sequence divergence is not a problem for the present methods, because some of the translocating and/or sequestering agents used in the present methods specifically bind to the HLH region. For example, the ENH protein, through its LIM domain, binds to the HLH region of Id proteins.

Id proteins are central to pathways regulating proliferation, differentiation, angiogenesis, migration, invasion and cell-cell interaction. Each pathway is important for the metastatic progression of cancer. Therefore, the present methods for sequestering Id proteins in the cytoplasm can serve to affect cancer progression by affecting one or more cellular signaling pathways important for proliferation, differentiation, angiogenesis, migration, invasion and cell-cell interaction, for example.

The present methods for treating cancer can also involve inhibiting the generation of blood vessels, because the generation of blood vessels is an essential step for the growth of primary and metastatic tumors. The generation of blood vessels, or neovascularization, increases the probability that tumor cells reach the bloodstream and colonize secondary sties. Mice deficient in both Id1 and Id3 show defective endothelial-cell vasculature in tumor xenografts and cannot support the growth or metastasis of three different tumors. But rather than targeting the expression of each Id protein, the present invention can provide methods for inhibiting angiogenesis by sequestering Id proteins in the cytoplasm with a common agent. In certain embodiments, the invention provides agents, such as LIM-domain comprising agents, that can bind to the HLH region of Id proteins. Because the HLH region is highly homologous between Id proteins, LIM-domain comprising agents can be used to sequester more than one Id protein in the cytoplasm of a cell.

### Translocating and/or Sequestering Agents

In certain embodiments, the methods for treating cancer, inhibiting angiogenesis or inhibiting proliferation of a tumor/cancer cell can comprise administering to a subject an agent that can sequester an Id protein(s) in the cytoplasm of a tumor/cancer cell. The agent can be, for example, a chemical compound, nucleic acid, protein or a peptide. In one embodiment, the agent can be a protein or peptide that comprises at least one LIM domain. In another embodiment, the agent can be a protein or peptide that comprises at least one PDZ domain. In another embodiment, the agent can be a protein or peptide that comprises at least one LIM domain and at least one PDZ domain. The PDZ and LIM domains contemplated by the invention can comprise amino acid sequences from naturally occurring PDZ-LIM proteins or mutants thereof. In one embodiment, an agent can be a recombinant protein or peptide that comprises a LIM domain that is encoded by a mixture of genetic sequences from more than one naturally occurring PDZ-LIM protein. In another embodiment, an agent can be a recombinant protein or peptide that comprises a PDZ domain that is encoded by a mixture of genetic sequences from more than one naturally occurring PDZ-LIM protein. In another embodiment, an agent can be a recombinant protein or peptide that comprises a PDZ domain and a LIM domain that are encoded by a mixture of genetic sequences from more than one naturally occurring PDZ-LIM protein. In one embodiment, the agent is a chemical compound that is a peptidomimetic, which mimics the ability of a LIM domain to specifically bind to an Id protein and/or mimics the ability of a PDZ domain to specifically bind to a cytoskeletal protien, including an actin protein. In one embodiment, the peptidomimetic mimics the ability of ENH to bind an Id protein and sequester it in the cytoplasm of a cell.

PDZ-LIM Family Proteins

ENH is a member of the Enigma family of LIM domain proteins. Proteins of the Enigma family possess an N-terminal PDZ domain and three LIM domains at the C-terminus (FIG. 1A). The LIM domain is a cysteine-rich double zinc finger motif, which mediates protein-protein interactions. All members of the PDZ-LIM Enigma family, including ENH, are cytoplasmic proteins that can associate with actin filaments through direct binding between the PDZ domain and α-actinin. Without being bound by theory, an Id protein can be sequestered in the cytoplasm through its interaction with the LIM domain of a PDZ-LIM family protein, and because the PDZ domain associates with the cytoskeleton of a cell through its association with actin filaments, the Id protein-PDZ-LIM-comprising protein complex is thereby sequestered in the cytoplasm.

PDZ-LIM proteins form a family based on single PDZ and LIM domains. Enigma represents the founding member of PDZ-LIM proteins, and was initially identified in a yeast two-hybrid screening as a LIM-domain containing protein that associates with the human insulin receptor (Wu and Gill, J. Biol. Chem., 269: 25085-25090 (1994)). Soon after the identification of Enigma several other related genes, including Ril (Kiess et al., Oncogene, 10: 61-68 (1995)), Clp-36 (Wang et al., Gene, 165: 267-271 (1995)) and ENH (Kuroda et al., J. Biol. Chem., 271: 31029-31032 (1996)) were identified through different approaches. From the discovery of ALP, it was revealed that these related genes encode proteins that not only have C-terminal LIM domains but also contain a highly similar N-terminal PDZ domain (Xia et al., J. Cell Biol., 139: 507-515 (1997)). The family now contains seven proteins (see FIG. 1), each of which contains one N-terminal PDZ domain followed either by one C-terminal LIM domain (ALP subfamily; ALP, RIL, CLP-36/hClim1/Elfin, Mystique) or three C-terminal LIM domains (Enigma subfamily; Enigma/LMP-1, ENH, ZASP/Cypher1) (Xia et al., 1997). The PDZ and LIM domains of these family members are highly related (up to 70% sequence similarity), whereas the middle part between PDZ and LIM domain is more diverse. In addition to ALP and Enigma subfamilies the human genome contains genes for three more distantly related proteins, namely LIMK-1 (Mizuno et al., Oncogene, 9:1605-1612 (1994)), LIMK-2 (Okano et al., J. Biol. Chem., 270: 31321-31330 (1995)), and LMO7 (Semenova et al., Hum. Mol. Genet., 12:1301-1312 (2003)), that each contains both PDZ and LIM domains. Proteins with PDZ and LIM domains or motifs are usually involved in developmental regulation, cellular differentiation and cytoskeleton organization. The PDZ-LIM family proteins can act as adapters that direct LIM-binding proteins, including the Id proteins, to the cytoskeleton.

Thus, in one embodiment, the invention provides methods for treating cancer (including inhibiting angiogenesis) by sequestering an Id protein(s) in the cytoplasm of a cell by expressing in the cell a PDZ-LIM protein that comprises at least one PDZ motif and at least one LIM domain. The PDZ-LIM protein can include, but is not limited to, ALP, RIL, CLP-36/hClim1/Elfin, Mystique, Enigma/LMP-1, ENH, ZASP/Cypher1, LIMK-1, LIMK-2, and LMO7. In one embodiment, the PDZ-LIM protein that is used in the present methods is a member of the ALP subfamily. In another embodiment, the PDZ-LIM protein that is used in the present methods is a member of the Enigma subfamily. In another embodiment, the PDZ-LIM protein that is used in the present methods is ENH. In another embodiment, the ENH protein can be any isoform of ENH, including but not limited to, isoform a (SEQ ID NO:4), b (SEQ ID NO:6), c (SEQ ID NO:8), d (SEQ ID NO:10), ore (SEQ ID NO:12) of human ENH. The invention encompasses mutants or variants of the above-mentioned PDZ-LIM proteins, including natural or man-made mutants or variants. In one embodiment, mutations are designed to increase the affinity of binding of a LIM domain to an Id protein, and/or to increase the affinity of binding the PDZ domain to the cytoskeleton (including actin filaments).

Recombinant Proteins Comprising PDZ and/or LIM Domains

In another embodiment, the present methods for treating cancer, inhibiting angiogenesis, etc., can comprise the use of a recombinant protein comprising at least one PDZ domain and/or at least one LIM domain. These recombinant proteins can comprise one or more PDZ domains from different proteins and/or one or more LIM domains from different proteins (or from one protein). Proteins comprising different combinations of LIM and PDZ domains can be generated by directed evolution methods and individual recombinant can be selected for by screening the ability of the recombinant proteins to sequester an Id protein in the cytoplasm. Alternatively, the recombinant proteins can be screened by testing their ability to inhibit angiogenesis, cellular proliferation, neoplastic transformation, or tumorigenesis.

By directed evolution, large numbers of different combinations of LIM domain, PDZ domain, and/or LIM and PDZ domains can be efficiently tested by protein domain shuffling or exon shuffling methods. In one embodiment, PDZ-LIM family members, either all homo sapiens or from any mammalian species, can be shuffled by DNA family shuffling (see Chang et al., Nat. Biotechnology, 17, 793-797 (1999)). DNA family shuffling is a method for permutation of natural genetic diversity. It is tool for rapidly evolving genes for desired properties. The method recombines more than two parental genes, or genes from different species, in a single DNA shuffling reaction (Stemmer, Biotechnology, 13, 549-555 (1995); Patten et al., Curr. Opin. Biotechnol., 8, 724-733 (1996); Crameri, Nature, 15, 288-291 (1998)). In brief, the method can comprise randomly fragmenting PDZ-LIM nucleic acids, for example, by using DNAse, and subjecting the fragments to recursive rounds of PCR. No exogenous primer needs to be added to the reaction, because fragments that possess regions of sequence homology to each other will serve to function as primers. Because the PDZ and LIM domains share sequence homology between family members within a species and between species, the requisite homology for priming exists. The PCR reaction will provide a population of recombinant sequences, wherein some of the recombinants will comprise PDZ and LIM domains that are composed of sequence blends from different family members. Additionally, DNA shuffling methods can be used with recombinant PDZ-LIM proteins as the starting material.

The PDZ (PSD-95, Dlg, ZO-1) domain was originally called a GLGF (Gly-Leu-Gly-Phe) repeat or a DHR region (discs large homology region). The human genome encodes for more than 400 PDZ domains in over 200 different proteins. These numbers indicate that frequently a single protein possesses more than one PDZ domain—sometimes even up to 13 as is the case with MUPP1. Another typical feature is that PDZ domains are grouped into pairs or triplets, and it has been proposed that these tandem PDZ domains might have an influence on protein folding and ligand binding. In several cases PDZ domains are found in combination with other domains, and depending on domain combinations PDZ domains have been classified into three main families; (1) proteins that contain only PDZ domains, (2) MAGUK PDZ proteins, which have one to three PDZ domains, a single SH3 domain, and a guanylate kinase domain, and (3) PDZ proteins that contain additional domains, such as LIM domains in the PDZ-LIM proteins. Numerous proteins containing PDZ domains localize to membrane structures, and are a part of bigger protein complexes such as membrane receptor complexes, ion channels, tight and adherens junctions. Therefore, without being bound by theory, recombinant proteins comprising just one or more PDZ domains can efficiently sequester Id proteins in the cytoplasm. In one embodiment, the invention provides methods for treating cancer, inhibiting angiogenesis, or inhibiting cell proliferation by expressing within a cell a protein comprising one or more PDZ domains and sequestering Id in the cytoplasm of the cell. In another embodiment, the protein can further comprise one or more LIM domains. The PDZ domain(s) can be from proteins that contain only PDZ domains, MAGUK PDZ proteins, or PDZ-LIM proteins.

PDZ domains that can be used in the invention include PDZ domains from the following list. In the following list, a Swiss-Prot database accession number for the protein is included, from which one skilled in the art can obtain the PDZ domain amino acid sequence if desired. Thus, proteins or protions thereof comprising a PDZ domain that can be used in the invention include, but are not limited to (GenBank Accession Nos. in parentheses), AFAD_HUMAN (P55196), AFAD_RAT (O35889), AIPI1_HUMAN (Q86UL8), AIP1_MOUSE (Q9WVQ1), AIP1_RAT (O88382), APBA1_HUMAN (Q02410), APBA1_RAT (O35430), APBA2_HUMAN (Q99767), APBA2_MOUSE (P98084), APBA2_RAT (O35431), APBA3_HUMAN (O96018), APBA3_MOUSE (O88888), APBA3_RAT (O70248), APXL_HUMAN (Q13796), ARHGB_HUMAN (O15085), ARHGB_RAT (Q9ES67), ARHGC_HUMAN (Q9NZN5), ARHGC_MOUSE (Q8R4H2), BAIP1_HUMAN (Q96QZ7), BAIP1_MOUSE (Q6RHR9), CAKI_DROME (Q24210), CAR14_HUMAN (Q9BXL6), CAR14_MOUSE (Q99KF0), CNKR1_HUMAN (Q969H4), CNKR2_HUMAN (Q8WXI2), CNKR2_MOUSE (Q80YA9), CNKR2_RAT (Q9Z1T4), CSKP_HUMAN (O14936), CSKP_MOUSE (O70589), CSKP_RAT (Q62915), CTPA_BARBA (Q44879), CTPA_SYNY3 (Q55669), DEGP1_ARATH (O22609), DEGP_BARHE (P54925), DEGP_BRUAB (P0A3Z6), DEGP_BRUME (Q8YG32), DEGP_BRUSU (P0A3Z5), DEGP_BUCAI (P57322), DEGP_BUCAP (O85291), DEGP_BUCBP (Q89AP5), DEGP_CHLMU (Q9PL97), DEGP_CHLPN (Q9Z6T0), DEGP_CHLTR (P18584), DEGP_ECOLI (P09376), DEGP_RHIME (Q52894), DEGP_RICCN (Q92JA1), DEGP_RICPR (O05942), DEGP_SALTY (P26982), DEGQ_ECOLI (P39099), DEGS_ECOLI (P31137), DEGS_HAEIN (P44947), DLG1_DROME (P31007), DLG1_HUMAN (Q12959), DLG1_MOUSE (Q811D0), DLG1_RAT (Q62696), DLG2_HUMAN (Q15700), DLG2_MOUSE (Q91XM9), DLG2_RAT (Q63622), DLG3_HUMAN (Q92796), DLG3_MOUSE (P70175), DLG3_RAT (Q62936), DLG4_HUMAN (P78352), DLG4_MOUSE (Q62108), DLG4_RAT (P31016), DLG5_HUMAN (Q8TDM6), DSH_DROME (P51140), DVL1L_HUMAN (P54792), DVL1_HUMAN (O14640), DVL1_MOUSE (P51141), DVL1_PANTR (Q5148), DVL1_RAT (Q9WVB9), DVL2_HUMAN (O14641), DVL2_MOUSE (Q60838), DVL2_XENLA (P51142), DVL3_HUMAN (Q92997), DVL3_MOUSE (Q61062), ECFE_ECOLI (P37764), ECFE_PHOLU (Q9S342), ECFE_SALTI (Q8Z9A4), ECFE_SALTY (Q8ZRP1), ECFE_YERPE (Q8ZH59), EEP_ENTFA (Q9RPP2), EM55_FUGRU (P49697), EM55_HUMAN (Q00013), EM55_MOUSE (P70290), ENH_HUMAN (Q96HC4), ENH_MOUSE (Q8CI51), ENH_RAT (Q62920), GIPC_HUMAN (O14908), GIPC_MOUSE (Q9Z0G0), GIPC_RAT (Q9Z254), GRIP1_HUMAN (Q9Y3R0), GRIP1_MOUSE (Q925T6), GRIP1_RAT (P97879), GRIP2_HUMAN (Q9C0E4), GRIP2_RAT (Q9WTW1), HTOA_HAEIN (P45129), HTRA1_HUMAN (Q92743), HTRA1_MOUSE (Q9R118), HTRA2_HUMAN (O43464), HTRA2_MOUSE (Q9JIY5), HTRA3_HUMAN (P83110), HTRA3_MOUSE (Q9D236), HTRA4_HUMAN (P83105), HTRA_BACSU (O34358), HTRA_LACHE (Q9Z4H7), HTRA_LACLA (Q9LA06), IL16_AOTTR (O62678), IL16_CERAE (O62674), IL16_HUMAN (Q14005), IL16_MACFA (O62676), IL16_MACMU (O62675), IL16_MOUSE (O54824), IL16_PANTR (O62666), IL16_SAISC (O62677), INAD_DROME (Q24008), LAP1_CAEEL (O61967), LAP1_DROME (Q9V780), LAP2_HUMAN (Q96RT1), LAP2_MOUSE (Q80TH2), LAP4_{—DROME (Q}7KRY7), LAP4_HUMAN (Q14160), LAP4_MOUSE (Q80U72), LIMK1_HUMAN (P53667), LIMK1_MOUSE (P53668), LIMK1_RAT (P53669), LIMK2_CHICK (P53666), LIMK2_HUMAN (P53671), LIMK2_MOUSE (O54785), LIMK2_RAT (P53670), LIN10_CAEEL (O17583), LIN2_CAEEL (P54936), LIN7A_HUMAN (O14910), LIN7A_MOUSE (Q8JZS0), LIN7A_RAT (Q9Z250), LIN7B_HUMAN (Q9HAP6), LIN7B_MOUSE (O88951), LIN7B_RAT (Q9Z252), LIN7C_HUMAN (Q9NUP9), LIN7C_MOUSE (O88952), LIN7C_RAT (Q79210), LMO7_HUMAN (Q8WWI1), LNX1_HUMAN (Q8TBB1), LNX1_MOUSE (O70263), LNX2_HUMAN (Q8N448), LNX2_MOUSE (Q91XL2), LRRC7_HUMAN (Q96NW7), LRRC7_MOUSE (Q80TE7), LRRC7_RAT (P70587), MAST1_HUMAN (Q9Y2H9), MAST1_MOUSE (Q9R1L5), MAST1_RAT (Q810W7), MAST2_HUMAN (Q6P0Q8), MAST2_MOUSE (Q60592), MPP2_HUMAN (Q14168), MPP2_MOUSE (Q9WV34), MPP3_HUMAN (Q13368), MPP3_MOUSE (O88910), MPP4_HUMAN (Q96JB8), MPP5_HUMAN (Q8N3R9), MPP5_MOUSE (Q9JLB2), MPP6_HUMAN (Q9NZW5), MPP6_MOUSE (Q9JLB0), MY18A_HUMAN (Q92614), MY18A_MOUSE (Q9JMH9), NEB1_HUMAN (Q9ULJ8), NEB1_RAT (O35867), NEB2_RAT (O35274), NHERF_HUMAN (O14745), NHERF_MOUSE (P70441), NHERF_RABIT (Q28619), NHERF_RAT (Q9JJ19), NHRF2_HUMAN (Q15599), NHRF2_MOUSE (Q9JHL1), NHRF2_RABIT (Q8SQG9), NHRF2_RAT (Q920G2), NOS1_HUMAN (P29475), NOS1_MOUSE (Q9Z0J4), NOS1_RABIT (O19132), NOS1_RAT (P29476), NOS1_SHEEP (Q29498), OXAA_RHOBA (Q7UFZ2), PAR3L_HUMAN (Q8TEW8), PAR6A_HUMAN (Q9NPB6), PAR6A_MOUSE (Q9Z101), PAR6B_HUMAN (Q9BYG5), PAR6B_MOUSE (Q9JK83), PAR6G_HUMAN (Q9BYG4), PAR6G_MOUSE (Q9JK84), PAR6_CAEEL (Q9NAN2), PARD3_CAEEL (Q17353), PARD3_HUMAN (Q8TEW0), PARD3_MOUSE (Q99NH2), PARD3_RAT (Q9Z340), PATJ_DROME (Q9NB04), PCLO_CHICK (Q9PU36), PCLO_MOUSE (Q9QYX7), PCLO_RAT (Q9JKS6), PDLI1_HUMAN (O00151), PDLI1_MOUSE (O70400), PDLI1_RAT (P52944), PDLI4_CHICK (Q9PW72), PDLI4_HUMAN (P50479), PDLI4_MOUSE (P70271), PDLI4_RAT (P36202), PDRN4_HUMAN (Q6ZMN7), PDZK3_HUMAN (O15018), PDZK3_RAT (Q9QZR8), PDZK4_HUMAN (Q76G19), PDZK4_MOUSE (Q9QY39), PICK1_HUMAN (Q9NRD5), PICK1_MOUSE (Q62083), PICK1_RAT (Q9EP80), PRAX_HUMAN (Q9BXM0), PRAX_MOUSE (O55103), PRAX_RAT (Q63425), PRC_ECOLI (P23865), PRC_HAEIN (P45306), PRC_SALTY (P43669), PREX1_HUMAN (Q8TCU6), PTN13_HUMAN (Q12923), PTN13_MOUSE (Q64512), PTN3_HUMAN (P26045), PTN4_HUMAN (P29074), PTP1_CAEEL (P28191), PZRN3_HUMAN (Q9UPQ7), PZRN3_MOUSE (Q69ZS0), PZRN3_RAT (P68907), RGS12_HUMAN (O14924), RGS12_RAT (O08774), RHPN1_HUMAN (Q8TCX5), RHPN1_MOUSE (Q61085), RHPN2_BRARE (Q6TNR1), RHPN2_CANFA (Q8HXG3), RHPN2_MOUSE (Q8BWR8), RIMS1_HUMAN (Q86UR5), RIMS1_RAT (Q9JIR4), RIMS2_HUMAN (Q9UQ26), RIMS2_MOUSE (Q9EQZ7), RIMS2_RAT (Q9JIS1), RIM_CAEEL (Q22366), RPGF2_HUMAN (Q9Y4G8), RPGF2_MOUSE (Q8CHG7), RPGF6_HUMAN (Q8TEU7), SDCB1_HUMAN (O00560), SDCB1_MOUSE (O08992), SDCB1_RAT (Q9JI92), SDCB2_HUMAN (Q9H190), SDCB2_MOUSE (Q99JZ0), SHAN1_HUMAN (Q9Y566), SHAN1_RAT (Q9WV48), SHAN2_HUMAN (Q9UPX8), SHAN2_RAT (Q9QX74), SHAN3_RAT (Q9JLU4), SI1L1_HUMAN (O43166), SI1L1_MOUSE (Q8C0T5), SI1L1_RAT (O35412), SI1L2_HUMAN (Q9P2F8), SI1L2_MOUSE (Q80TE4), SI1L3_HUMAN (O60292), SIF1_DROME (P91621), SIF2_DROME (P91620), SIPA1_HUMAN (Q96FS4), SIPA1_MOUSE (P46062), SLIP1_DROME (Q8MR31), SNT1_CAEEL (Q93646), SNTA1_HUMAN (Q13424), SNTA1_MOUSE (Q61234), SNTA1_RABIT (Q28626), SNTB1_HUMAN (Q13884), SNTB1_MOUSE (Q99L88), SNTB2_HUMAN (Q13425), SNTB2_MOUSE (Q61235), SNTG1_HUMAN (Q9NSN8), SNTG1_MOUSE (Q925E1), SNTG2_HUMAN (Q9NY99), SNTG2_MOUSE (Q925E0), SYJ2B_HUMAN (P57105), SYJ2B_RAT (Q9WVJ4), SYNP2_HUMAN (Q9UMS6), SYNP2_MOUSE (Q91YE8), SYP2L_HUMAN (Q9H987), SYP2L_MOUSE (Q8BWB1), TIAM1_HUMAN (Q13009), TIAM1_MOUSE (Q60610), USH1C_HUMAN (Q9Y6N9), USH1C_MOUSE (Q9ES64), WHRN_HUMAN (Q9P202), WHRN_MOUSE (Q80VW5), WHRN_RAT (Q810W9), Y084_NEIMA (Q9JX32), Y1047_XYLFA (Q9PEI1), Y1156_BRUSU (Q8G0E1), Y1318_LISMO (Q8Y7G3), Y1355_LISIN (Q92C36), Y1380_{—AGRT}5 (Q8UFL7), Y1411_RALSO (Q8XZI4), Y1507_DEIRA (Q9RU85), Y161_RICPR (Q9ZE02), Y1693_CLOPE (Q8XJR2), Y183_NEIMB (Q9K1G9), Y1964_AQUAE (O67776), Y203_RICCN (Q92J66), Y2128_LACLA (Q9CDT3), Y2253_VIBCH (Q9KPV9), Y2420_BACHD (Q9KA70), Y2869_MYCTU (O33351), Y327_XYLFT (Q87EI0), Y3971_ANASP (Q8YQ64), Y638_RHILO (Q98MC1), Y829_BRUME (Q8YHH1), Y890_THEMA (Q9WZZ2), Y938_SYNP6 (P05676), YPEB_SYNP2 (P42784), YQI7_CAEEL (Q09506), YQJ3_CAEEL (Q09284), YTE4_CAEEL (Q17865), YVTA_BACSU (Q9RI91), ZO1_HUMAN (Q07157), ZO1_MOUSE (P39447), ZO2_CANFA (Q95168), ZO2_HUMAN (Q9UDY2), ZO2_MOUSE (Q9Z0U1), ZO3_CANFA (O62683), ZO3_HUMAN (O95049), and ZO3_MOUSE (Q9QXY1).

In one embodiment, a PDZ domain of the invention can comprise, for example, a sequence that is at least 75%, 80%, 85%, 90%, 95%, or 99% identical to: residues 10-82 of SEQ ID NO:4; residues 10-82 of SEQ ID NO:6; residues 10-82 of SEQ ID NO:6; residues 10-82 of SEQ ID NO:8; residues 10-82 of SEQ ID NO:10; or residues 10-82 of SEQ ID NO:12.

The LIM domain is a 50-60 amino acid residue double zinc-finger motif, which was initially identified as a cysteine-rich domain in three different transcription factors (Lin-11, Isl-1, mec-3; reviewed in; Brown et al., 2001; Khurana et al., 2002). LIM domains can be found from a variety of proteins including transcription factors, cytoskeletal proteins and signaling molecules. LIM domain contains conserved cysteine, histidine, and aspartic acid residues, which are important for zinc binding whereas the rest of the amino acids in this domain are variable, and apparently involved in interaction with target protein. LIM domains can have the following amino acid consensus sequence: C-x(2)-C-x(15,21)-[FYWHPCR]-H-x(2)-[CH]-x(2)-C-x(2)-C-x(3)-[LIVMF] (SEQ ID NO:2; Prosite Accession No. PS00478). The “x” represents any amino acid and the numbers represent the number of amino acids at that location. The “(15, 21)” indicates that there can be either 15 amino acids or 21 amino acids at that location.

LIM domain that can be used in the invention include, but are not limited, the LIM domains of the following list of proteins. The LIM domains that are present in the following proteins can be ascertained to one skilled in the art by reference to the accession number provided in the list below. Alternatively, one skilled in the art can determine from the amino acid sequence of the proteins where the LIM domain is present by identifying the amino acid sequence that matches the LIM domain consensus sequence described above. Thus, the LIM domain comprising proteins include, but are not limited to (GenBank Accession Nos. in parentheses): ABLM1_HUMAN (O14639), ABLM1_MOUSE (Q8K4G5), ABLM2_HUMAN (Q6H8Q1), ABLM2_MOUSE (Q8BL65), ABLM2_RAT (Q6KC51), ABLM3_HUMAN (O94929), ABLM3_MOUSE (Q69ZX8), APTE_DROME (P29673), AWH_DROME (Q8IRC7), CRIP1_HUMAN (P50238), CRIP1_MOUSE (P63254), CRIP1_RAT (P63255), CRIP2_HUMAN (P52943), CRIP2_MOUSE (Q9DCT8), CRIP2_RAT (P36201), CSRP1_CHICK (P67966), CSRP1_COTJA (P67967), CSRP1_HUMAN (P21291), CSRP1_MOUSE (P97315), CSRP1_RAT (P47875), CSRP2_CHICK (P50460), CSRP2_COTJA (Q05158), CSRP2_HUMAN (Q16527), CSRP2_MOUSE (P97314), CSRP2_RAT (Q62908), CSRP3_HUMAN (P50461), CSRP3_MOUSE (P50462), CSRP3_RAT (P50463), ENH_HUMAN (Q96HC4), ENH_MOUSE (Q8CI51), ENH_RAT (Q62920), EPLIN_HUMAN (Q9UHB6), EPLIN_MOUSE (Q9ERG0), FBLP1_HUMAN (Q8WUP2), HM14_CAEEL (P20271), ISL1_BRARE (P53405), ISL1_CHICK (P50211), ISL1_HUMAN (P61371), ISL1_MESAU (P61373), ISL1_MOUSE (P61372), ISL1_RAT (P61374), ISL2A_ONCTS (P53408), ISL2B_ONCTS (P50212), ISL2_{—BRARE (P}53406), ISL2_CHICK (P53410), ISL2_HUMAN (Q96A47), ISL2_MOUSE (Q9CXV0), ISL2_RAT (P50480), ISL3_BRARE (P53407), ISL3_ONCTS (P53409), LASP1_CAEEL (P34416), LASP1_DROME (Q817C3), LASP1_HUMAN (Q14847), LASP1_MOUSE (Q61792), LASP1_PIG (P80171), LASP1_RAT (Q99MZ8), LHX1_BRARE (Q90476), LHX1_CHICK (P53411), LHX1_HUMAN (P48742), LHX1_MESAU (P63008), LHX1_MOUSE (P63006), LHX1_PANTR (Q5IS44), LHX1_RAT (P63007), LHX1_SAIBB (Q5IS89), LHX1_XENLA (P29674), LHX2_HUMAN (P50458), LHX2_MOUSE (Q9ZOS2), LHX2_RAT (P36198), LHX3_BRARE (Q90421), LHX3_CHICK (P53412), LHX3_HUMAN (Q9UBR4), LHX3_MOUSE (P50481), LHX3_PIG (O97581), LHX3_XENLA (P36200), LHX4_HUMAN (Q969G2), LHX4_MOUSE (P53776), LHX5_BRARE (P52889), LHX5_HUMAN (Q9H2C1), LHX5_MOUSE (P61375), LHX5_RAT (P61376), LHX5_XENLA (P37137), LHX61_HUMAN (Q9UPM6), LHX61_MOUSE (Q9R1R0), LHX6_MOUSE (O88706), LHX8_MOUSE (O35652), LHX9_HUMAN (Q9NQ69), LHX9_MOUSE (Q9WUH2), LIMD1_HUMAN (Q9UGP4), LIMD1_MOUSE (Q9QXD8), LIMK1_HUMAN (P53667), LIMK1_MOUSE (P53668), LIMK1_RAT (P53669), LIMK2_CHICK (P53666), LIMK2_HUMAN (P53671), LIMK2_MOUSE (O54785), LIMK2_RAT (P53670), LIM_HALRO (Q25132), LIN11_CAEEL (P20154), LMCD1_HUMAN (Q9NZU5), LMCD1_MOUSE (Q8VEE1), LMO3_HUMAN (Q8TAP4), LMO3_MOUSE (Q8BZL8), LMO3_RAT (Q99MB5), LMO4_HUMAN (P61968), LMO4_MOUSE (P61969), LMO6_HUMAN (O43900), LMO7_HUMAN (Q8WWI1), LMX1A_HUMAN (Q8TE12), LMX1A_MESAU (Q04650), LMX1A_MOUSE (Q9JKU8), LMX1B_CHICK (P53413), LMX1B_HUMAN (O60663), LMX1B_MESAU (Q60564), LMX1B_MOUSE (O88609), LPXN_HUMAN (O60711), LRG1_YEAST (P35688), MEC3_CAEBR (P34764), MEC3_CAEEL (P09088), MEC3_CAEVU (P34765), MICA1_HUMAN (Q8TDZ2), MICA1_MOUSE (Q8VDP3), MICA2_HUMAN (O94851), MICA2_MOUSE (Q8BML1), MICA3_HUMAN (Q7RTP6), MICA3_MOUSE (Q8CJ19), MILK1_HUMAN (Q8N3F8), MILK1_MOUSE (Q8BGT6), MILK2_HUMAN (Q8IY33), MLP1_DROME (P53777), MLP2_DROME (Q24400), PAXI_CHICK (P49024), PAXI_HUMAN (P49023), PDLI1_HUMAN (O00151), PDLI1_MOUSE (O70400), PDLI1_RAT (P52944), PDLI4_CHICK (Q9PW72), PDLI4_HUMAN (P50479), PDLI4_MOUSE (P70271), PDLI4_RAT (P36202), PIN2_CAEEL (Q19157), PINC_HUMAN (P48059), PRIC1_HUMAN (Q96MT3), PRIC1_RAT (Q71QF9), PRIC2_HUMAN (Q7Z3G6), PRIC2_MOUSE (Q80Y24), RBTN1_HUMAN (P25800), RBTN1_MOUSE (Q924W9), RBTN2_HUMAN (P25791), RBTN2_MOUSE (P25801), RGA1_SCHPO (O43052), RGA1_YEAST (P39083), RGA2_YEAST (Q06407), RGA3_SCHPO (O14014), RGA4_SCHPO (O74360), SCEL_HUMAN (O95171), SCEL_MOUSE (Q9EQG3), SLIM1_HUMAN (Q13642), SLIM1_MOUSE (P97447), SLIM2_HUMAN (Q13643), SLIM2_MOUSE (Q9R059), SLIM3_HUMAN (Q14192), SLIM3_MOUSE (O70433), SLIM3_RAT (O35115), TES_HUMAN (Q9UGI8), TES_MOUSE (P47226), TRIP6_HUMAN (Q15654), TSF3_HELAN (P29675), UNC97_CAEEL (P50464), YK70_YEAST (P36166), YP96_CAEEL (Q09476), ZN185_HUMAN (O15231), ZN185_MOUSE (Q62394), ZYX_CHICK (Q04584), ZYX_HUMAN (Q15942), and ZYX_MOUSE (Q62523).

In one embodiment, a LIM domain of the invention can comprise, for example, a sequence that is at least 75%, 80%, 85%, 90%, 95%, or 99% identical to: residues 420-470 of SEQ ID NO:4; residues 479-529 of SEQ ID NO:4; residues 537-590 of SEQ ID NO:4; residues 311-361 of SEQ ID NO:6; residues 370-420 of SEQ ID NO:6; or residues 428-481 of SEQ ID NO:6.

LIM domains can regulate the subcellular localization of interacting proteins. In one embodiment, the invention provides methods for treating cancer, inhibiting angiogenesis, or inhibiting cell proliferation by expressing within a cell a protein comprising one or more LIM domains and sequestering Id in the cytoplasm of the cell. In another embodiment, the protein can further comprise one or more PDZ domains.

The majority of PDZ-LIM proteins have been shown to associate with the actin cytoskeleton, suggesting that their functions are related. PDZ-LIM proteins associate on one hand with the actin cytoskeleton via their PDZ domain, and on the other hand with kinases via their LIM domain. The present invention provides the discovery that Id proteins can bind to PDZ-LIM proteins, thus, it is possible that PDZ-LIM proteins can associate on the one hand with the actin cytoskeleton via their PDZ domain, and on the other hand with Id proteins via their LIM domain. Without being bound by theory, it is possible that this dual-function of PDZ-LIM proteins can serve to sequester Id proteins in the cytoplasm. However, it is possible that proteins containing just a LIM domain can sequester an Id protein in the cytoplasm, for example, by preventing an Id protein to freely diffuse into the nucleus, i.e., the LIM-Id protein complex can have an aggregate size (about greater than 40 kilodaltons) that is too large to pass through a nuclear pore complex. Thus, in certain embodiments, the invention provides methods for treating cancer, inhibiting angiogenesis, or inhibiting cell proliferation that comprises expressing a protein comprising a LIM domain(s), wherein the protein does not comprise a PDZ domain, and sequestering an Id protein in the cytoplasm of the cell.

### Delivery of Sequestering and/or Translocating Agents

The PDZ/LIM proteins or peptides of the invention can be introduced into a cell as a protein or as a nucleic acid encoding that protein or peptide. When a protein or peptide is used, it can optionally have a cellular-uptake signal to facilitate uptake of the protein by the cell. The amount of the protein or peptide needed to sequester an Id protein in the cytoplasm can be readily determined by those of skill in the art, for example, by testing different amounts sufficient to inhibit Id-mediated cellular proliferation in vitro (see Examples for Id-mediated cellular proliferation assays). Further, the amount of an expression vector (that expresses a protein or peptide agent) needed to sequester an Id protein in the cytoplasm such that cell proliferation is inhibited can be readily determined by those of skill in the art.

When a nucleic acid such as RNA or DNA is used that encodes a protein or peptide of the invention, it can be delivered into a cell in any of a variety of forms, including as naked plasmid or other DNA, formulated in liposomes, in an expression vector, which includes a viral vector (including RNA viruses and DNA viruses, including adenovirus, alphavirus, and adeno-associated virus), by biocompatible gels, via a pressure injection apparatus such as the Powdererjet™ system using RNA or DNA, or by any other convenient means. Again, the amount of nucleic acid needed to sequester an Id protein in the cytoplasm can be readily determined by those of skill in the art, which also may vary with the delivery formulation and mode and whether the nucleic acid is DNA or RNA.

Administration

When administered to a subject, an agent of the invention can be administered as a component of a composition that comprises a physiologically acceptable carrier or vehicle. When the agent comprises a chemical compound, it can be administered orally. The agents of the invention can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc. including the delivery systems mentioned in the prior section for proteins and peptides, and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the agents into the bloodstream. The mode of administration is left to the discretion of the practitioner.

In one embodiment, the agents are administered orally. In another embodiment, the agents are administered intravenously. In other embodiments, it can be desirable to administer the agents locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the agents into the cardiovascular system, pulmonary system, lymphatic system, central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir (a device implanted under the scalp that is used to deliver anticancer drugs to the cerebrospinal fluid, the fluid surrounding the brain and spinal cord).

Pulmonary administration can also be employed, e.g., by use of an inhaler of nebulizer, and formulation with an aerosolizing agent, or via perfusion in a fluorocarbon or a synthetic pulmonary surfactant. In certain embodiments, the agents can be formulated as a suppository, with traditional binders and excipients such as triglycerides.

In another embodiment the agents can be delivered in a vesicle, in particular a liposome (see Langer, Science 249:1527-1533 (1990)).

In yet another embodiment, the agents can be delivered in a controlled-release system or sustained release system (see, e.g., Goodson, in Medical Applications of Controlled Release, CRC Press, Bocca Raton, Fla., vol. 2, pp. 115-138 (1984)). Other controlled or sustained release systems discussed in the review by Langer (Science 249:1527-1533 (1990)) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1985); During et al., Ann. Neural. 25:351 (1989); and Howard et al., J. Neurosurg. 71:105 (1989)).

In yet another embodiment, a controlled or sustained release system can be placed in proximity of a target tissue (including the tumor tissue) of the agent, e.g., the heart, spinal column, brain, skin, lung, or gastrointestinal tract, thus requiring only a fraction of the systemic dose. In one embodiment, the controlled or sustained release system comprises a biodegradable gel that carries an agent of the invention, such that the gel can be implanted into a region near or in tumor tissue. In one embodiment, the biodegradable gel carries an agent that inhibits angiogenesis by sequestering an Id protein(s) in the cytoplasm of a cell.

The present compositions can optionally comprise a suitable amount of a physiologically acceptable excipient so as to provide the form for proper administration to the subject.

Such physiologically acceptable excipients can be liquids, such as water and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water can be a useful excipient when the agent is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present agents can take the form of solutions, suspensions, emulsion, tablets, pills; pellets, capsules, capsules containing liquids, powders, sustained release formulations, suppositories, emulsions, aerosols, sprays, suspensions, or any other form suitable for use. In one embodiment, the agent is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment, the agents are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more non-active agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving an agent (as used herein, an “agent” refers to an entity that can sequester an Id protein(s) in the cytoplasm of a cell; where the invention refers to agents that are not the active ingredient for the methods of treatments mentioned herein, then the agent is referred to as an “inactive-agent”) are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment, the agents can be formulated for intravenous administration. Typically, compositions for intravenous administration comprise sterile isotonic aqueous buffer. If the agent is a nucleic acid, then the compositions for intravenous administration (and other routes and methods of administration) can comprise an aqueous buffer that contains necessary salts to maintain nucleic acid stability. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the agents are to be administered by infusion, they can be dispensed, for example, with an infusion bottle containing sterile pharmaceutical grade water or saline. Where the agents are administered by injection, an ampule of sterile water for injection or saline can be provided so that the ingredients can be mixed prior to administration.

The agents can be administered by controlled-release or sustained release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled or sustained release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled or sustained release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled or sustained release.

The amount of the agent that is effective: (i) in the treatment or prevention of a condition; (ii) to translocate and/or sequester an Id protein(s) to/in the cytoplasm of cells in a tumor or other tissue; (iii) to inhibit cell proliferation, (iv) to promote cell differentiation, or (v) to inhibit angiogenesis in a subject, can be determined by standard clinical techniques. In addition, in vitro or in vivo assays can optionally be employed to help identify optimal dosage ranges. The precise dose to be employed will also depend on the route of administration, and the seriousness of the condition being treated and should be decided according to the judgment of the practitioner and each subject's circumstances in view of, e.g., published clinical studies. Suitable effective dosage amounts of an agent that is a chemical compound, however, can range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about I g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. Suitable effective dosage amounts of an agent that is a protein, peptide or nucleic acid can be based on the effective dosage amounts suggested above for a chemical compound.

The agents can be assayed in vitro or in vivo for the desired therapeutic or prophylactic activity prior to use in humans. A exemplary in vitro model is described in Example 2 (assessing whether an agent can inhibit Id-mediated cellular proliferation); further, methods of screening are described below. Subject model systems can be used to demonstrate safety and efficacy.

### Methods of Screening

The invention also provides methods for screening whether an agent can sequester/translocate an Id protein in/to the cytoplasm of a cell and methods for screening whether an agent can inhibit proliferation of a cell. If the agent can sequester/translocate an Id protein in/to the cytoplasm or inhibit proliferation, then the agent is considered to be a potential drug for treating cancer.

Methods for Screening Id Cytoplasmic Sequestration or Retention

In certain embodiments, methods for screening whether an agent can sequester an Id protein in the cytoplasm can comprise immunofluorescence. In one embodiment, a cell that endogenously expresses an Id protein is transfected, transiently or stably, with an expression vector that expresses a protein that comprises at least one LIM domain and/or one PDZ domain. To verify whether the LIM/PDZ protein is expressed in the cell, RT-PCR assays or Western blots can be conducted. Western blots can be conducted by including an epitope coding region in the coding sequence of the LIM/PDZ protein. The epitope can be, for example, an HA-tag. Prior to ectopic expression of the LIM/PDZ protein, the cell should be examined to determine that the Id protein under investigation is localized to the nucleus. After transfection, the cell is then assayed with labeled antibodies that are specific to an Id protein to determine whether the Id proteins in the cell are predominantly localized in the cytoplasm. If the Id protein in the cell is localized in the cytoplasm, then the LIM/PDZ protein is considered to be an agent that can sequester and/or translocate an Id protein in/to the cytoplasm.

In one embodiment, a chemical compound agent is added to a cell, where it is known that an Id protein is localized to the nucleus. The Id protein can be endogenously expressed by the cell or ectopically expressed (for example, by an expression vector that is transiently or stably transfected into the cell). After addition of the chemical compound to the cell, immunofluorescence is conducted to determine whether the Id protein has a cytoplasmic subcellular localization. If the Id protein in the cell is localized in the cytoplasm, then the chemical compound is considered to be an agent that can sequester and/or translocate an Id protein in/to the cytoplasm.

In another embodiment, methods for screening whether an agent can sequester an Id protein in the cytoplasm involves assaying for co-localized fluorescence from an Id reporter fusion protein and a cytoskeletal reporter fusion protein. For example, fluorescent reporter fusion proteins can be made with an Id protein fused to a fluorescent protein and a cytoskeletal protein, such as actin, fused to a fluorescent protein. The fluorescent proteins include, but are not limited to, red fluorescent protein (RFP), yellow fluorescent protein (YFP), cyan fluorescent protein (CFP), green fluorescent protein (GFP), blue fluorescent protein (BFP), sapphire fluorescent protein, and their variants. The Id fusion reporter protein and the cytoskeletal reporter fusion protein should comprise different fluorescent proteins. Thus, a cell is first transfected, stably or transiently, with a cytoskeletal reporter fusion protein to determine whether this fusion protein integrates into the cytoskeleton of the cell. If integration is successful, then the Id fusion reporter protein is transfected into the cell to verify that this fusion protein localizes to the nucleus. If the Id fusion reporter protein localizes to the nucleus, then the cell can then be transfected with an expression vector encoding a LIM/PDZ protein or incubated with a chemical compound to determine whether the Id fusion protein then obtains a cytoplasmic subcellular localization.

The subcellular localization of the Id fusion protein can be determined in a high-throughput manner by confocal fluorescence coincidence analysis. Confocal fluorescence coincidence analysis extracts fluorescence fluctuations that occur coincidently in two different spectral ranges. This procedure makes it possible to monitor whether an association between molecular fragments that are labeled with different fluorophores is established or broken. Confocal fluorescence coincidence analysis is a very sensitive and ultrafast technique with readout times of 100 ms and below. Thus, if the Id fusion protein localizes in the cytoplasm, the confocal fluorescence coincidence analysis should detect fluorescence from both the Id fusion reporter protein and the cytoskeletal fusion reporter protein. If colocalized fluorescence is detected, then this indicates that the LIM/PDZ protein can sequester an Id protein in the cytoplasm.

Alternatively, agents can be tested with the use of an Id fusion reporter protein alone. After verifying that the Id fusion reporter protein has a nuclear localization, an agent is added to the cell. The cell can then be stained with DAPI (4′,6-diamidino-2-phenylindole), which specifically stains the nucleus of cells. If the agent causes the Id fusion reporter protein to translocate to, or be sequestered in, the cytoplasm, then fluorescent microscopy should show on image overlays that the fluorescence of the Id fusion reporter protein is not coincident with DAPI fluorescence.

Alternatively, the subcellular localization of the Id fusion protein can be determined in a high-throughput manner by fluorescene resonance emission transfer (FRET) detection or time-resolved FRET (TR-FRET) detection. FRET has been established as a sensor of protein-protein interactions (Miyawaki et al. (1997), Nature, 388: 882-887). When two proteins comprising fluorescent protein domains interact, exciting light causes one of the fluorescent protein domains (fluorophore donor) to transfer its energy to a second, longer wavelength fluorophore (fluorophore acceptor) in a non-radiative manner. When there is a transfer of excitation energy from the donor to the acceptor, there is no emission by the donor. Therefore, when a LIM/PDZ protein can cause an Id protein to be sequestered in, and/or translocated to, the cytoplasm, the Id fusion protein and the cytoskeletal fusion protein can interact such that energy is transferred, resulting in no emission by the donor, and thus, resulting in the emission of only one type of fluorescence. However, if the Id reporter fusion protein does not obtain a cytoplasmic subcellular localization, then two types of fluorescence are detected. Fluorescence energy emission transfer is distance dependent; two fluorophores have to be in sufficient proximity (less than 100 angstroms) such that an excited fluorophore can transfer its energy to a second fluorophore. Therefore, if one only type of fluorescence is detected, then this indicates that the agent can sequester Id in the cytoplasm.

In another embodiment, a method for screening can comprise bioluminescence resonance energy transmission (BRET). In BRET, there is a luminescent donor protein and a fluorescent acceptor protein. For example, the luminescent donor protein can comprise an Id protein fused to luciferase and the fluorescent acceptor protein can comprise an actin protein or domain fused to yellow fluorescent protein. In most applications the fused donor is Renilla luciferase (Rluc) and the acceptor is a fusion with YFP (to increase the spectral distinction between the two emissions). When the donor and acceptor are in close proximity, i.e., when the two fusion proteins interact, the energy resulting from catalytic degradation of a coelenterazine derivative substrate is transferred from the luciferase to the YFP, which will then emit fluorescence at its characteristic wavelength. Thus, when the two fusion proteins interact, there are two signals, luciferase and YFP. When the two fusion proteins do not interact, for example, when an agent cannot cause the Id fusion protein to be sequestered in and/or to be translocated to the cytoplasm, then there is only one signal that can be detected, the excited luciferase.

Methods for Screening Inhibition of Id-Mediated Tumor Growth

The invention provides methods for screening agents to determine whether the agents can inhibit tumor growth. In certain embodiments, the screening methods relate to whether an agent can prevent Id-mediated cellular proliferation. In certain embodiments, the screening methods relate to whether an agent can reduce or prevent tumor growth in a subject. In other embodiments, the screening methods relate to whether an agent can prevent angiogenesis in tumor tissue.

An agent can be screened to determine whether it can prevent Id-mediated cellular proliferation by testing whether the agent can inhibit Id-2 mediated stimulation of DNA synthesis, which can be performed by transfecting different combinations of expression vectors into a cell. For example, (see Example 2) cells can be transfected with: (a) a negative control comprising an “empty vector” or an expression vector that does not comprise a coding region for a protein; (b) an expression vector encoding an Id protein; and (c) an expression vector encoding an Id protein and an expression vector encoding a PDZ/LIM protein. Further, to test a chemical compound agent, the chemical compound can be added to cell cultures that have been transfected with an expression vector encoding an Id protein. For these experiments, an expression vector expressing a reporter gene, such as GFP, which provides an indicator to determine which cells are transfected—because the principle of co-transfection provides that multiple plasmids can be transfected into a single cell. After transfection, cell cultures are labeled with BrdU and immunostained for BrdU using a primary antibody specific to BrdU and a secondary antibody conjugated with a fluorophore, such as Cy3. Cells are then assessed for GFP and BrdU, and the percentage of transfected cells positive for BrdU are scored. When the percentage of transfected cell positive for BrdU is reduced (in cell cultures that have been transfected with an Id expression vector and a PDZ-LIM expression vector or in cell cultures that have been transfected with an Id expression vector and incubated with a chemical compound agent) as compared to control experiments, this indicates that the agent can inhibit Id mediated cellular proliferation. Exemplary cell lines that can be used include, but are not limited to, the glioma cell line SF188 and the neuroblastoma cell lines IMR-32, SK-N-SH, SH-F and SH-N.

To test agents of the invention for their ability to treat tumors in vivo, tumors can be explanted into nude mice (i.e., athymic mice). After the tumors are established in the mice, the agents contemplated by the invention can be administered to the mice in order to test for whether the agents can diminish the tumors. An exemplary tumor cell line that can be explanted into the nude mice is the SH-N-Flag-Id2 cell line (or a SH-N-Flag-Id1 (or -Id3, or -Id4) cell line) that expresses an Id protein that is localized to the nucleus. Other tumor cell lines that stably express an Id protein can also be used. These Id-expressing cell lines can provide tumor mouse models. To test whether a PDZ-LIM can prevent tumor growth, the tumor cell line that expresses the Id protein is stably transfected to also express a LIM/PDZ protein. If a LIM/PDZ protein can sequester Id in the cytoplasm, then it is expected that the LIM/PDZ protein can oppose some or all Id-mediated activities that stimulate tumor growth, including neoangiogenesis. To test whether a PDZ-LIM protein or a chemical compound agent can reduce or inhibit tumor growth, these agents can be administered to a mouse comprising an explanted tumor at any stage of tumor growth to assess whether the presence of the agent (or dose or route of administration) is effective to reduce tumor size or prevent further growth of the tumor.

To assess whether an agent inhibits angiogenesis, the presence of an agent should result in impaired expression of VEGF with disrupted vascularity in the tumors. Tumor angiogenesis in mouse tumor models can be examined at a macroscopic (through injectin of Orange microfil latex) and at a microscopic level (expression of markers for new tumor blood vessels such as PECAM and SMA) (Lasorella, A. et al., “Id2 mediates tumor initiation, proliferation and angiogenesis in Rb mutant mice,” Mol. Cell. Biol., submitted). The expression of VEGF can be scored by immunohistochemistry and western blot analysis of the different tumors.

### Additional Embodiments

In one embodiment, the invention provides methods for grading a tumor. Id proteins can contribute to the proliferation of tumor cells, i.e., contribute to the neoplastic nature of a tumor, if they are functioning in the nucleus. However, once neoplastic cells begin to differentiate, Id proteins may be localized in the cytoplasm, because it may be necessary for Id function to be repressed or blocked in order for the cell to differentiate. Without being bound by theory, tumor cells might sometimes express PDZ-LIM proteins to block Id-mediated cell proliferation in order to differentiate. If a PDZ-LIM protein is naturally expressed in a tumor cell, this can indicate that the tumor cell has already proliferated and has now differentiated into a different stage. Thus, in one embodiment, the invention provides methods comprising determining whether a native/endogenous PDZ-LIM protein member is expressed in a tumor cell, wherein if the PDZ-LIM protein is expressed then the tumor is graded higher or as a more aggressive tumor than if the PDZ-LIM protein is not expressed.

In one embodiment, the invention provides devices that are coated with or carry or otherwise contain an agent(s) contemplated herein, including a PDZ/LIM protein or a chemical compound. Again, the agents contemplated herein are related with respect to their ability to sequester an Id protein in the cytoplasm of the cell. Therefore, devices coated with or carrying such agents can be implanted into subjects in order to provide a localized administration of the agents. A localized administration of the agents can be useful against tumor tissue, such that the localized administration can provide a higher dose of the agents to target cells. Thus, in one embodiment, the invention provides a medical device comprising a coating of a protein comprising a LIM domain, or a chemical compound, wherein the protein or the chemical compound sequesters an Id protein in the cytoplasm of a cell. In another embodiment, the medical device is a stent coated with ENH proteins. Such a stent can be useful in inhibiting angiogenesis in tumor tissue, i.e., the stent can be placed in existing tumor vascularizations, such that the new blood vessel formation is prevented.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### Tumor Targets

The invention can be used to treat various tumors/cancers, including but not limited to, small cell carcinoma (SCC) of the head, neck, esophagus and oral cavity; melanoma; hepatocellular carcionoma; colorectal adenocarcinoma; pancreatic cancer; medullary thyroid cancer; papillary thryoid cancer; astrocytic tumor; neuroblastoma; Ewing's sarcoma; ovarian tumor; cervical cancer; endometrial carcinoma; breast cancer; prostate cancer; nervous system tumors; Wilms' tumor; retinoblastoma; leukemia; and malignant seminoma. The central nervous system tumor can include, but is not limited to, cerebellar astrocytoma, medulloblastoma, ependymona, brain stem glioma, optic nerve glioma, or germinoma. In one embodiment, the methods for treating cancer relate to methods for inhibiting proliferation of a cancer or tumor cell comprising administering to a subject a protein or other agent that sequesters an Id protein(s) in the cytoplasm of the tumor or cancer cell. The table below is a summary of the correlation between some of these tumor types and altered expression levels of Id proteins.

TABLE 1
Tumor Types and Altered Id Expression Levels

| Tumor Type | Id Protein | Id Expression Level |
|---|---|---|
| Squamous cell carcinoma | Id1, Id2, Id3 | Elevated |
| (SCC), head and neck | | |
| SCC, esophageal | Id1 | Elevated |
| SCC, oral cavity | Id1 | Elevated |
| Melanoma | Id1 | Elevated |
| Hepatocellular carcinoma | Id1 | Elevated |
| Colorectal adenocarcinoma | Id1, Id2, Id3 | Elevated |
| Pancreatic cancer | Id1, Id2 | Elevated |
| Medullary thyroid cancer | Id1 | Elevated |
| Papillary thyroid cancer | Id1 | Elevated |
| Astrocytic tumor | Id1, Id2, Id3 | Elevated |
| Neuroblastoma | Id2 | Elevated |
| Ewing's sarcoma | Id2 | Elevated |
| Ovarian tumor | Id1 | Elevated |
| Ovarian tumor | Id3 | Under expressed in 70% |
| | | of ovarian tumors |
| Cervical cancer | Id1 | Elevated |
| Endometrial carcinoma | Id1 | Elevated |
| Breast cancer | Id1, Id3 | Elevated |
| Breast cancer | Id2 | Decreased levels that |
| | | negatively correlate with |
| | | cancer progression |
| Prostate cancer | Id1, Id2 | Elevated |
| Malignant seminoma | Id1, Id2, Id3, Id4 | Elevated |

### EXAMPLES

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the examples described below are provided to illustrate the present invention and are not included for the purpose of limiting the invention.

### Example 1

### ENH is a Cytoplasmic Sequestration Factor for ID2 in Normal and Tumor Cells from the Nervous System

The data in this Example indicates that ENH can extinguish Id2 function and initiate bHLH-mediated transcription, therefore allowing differentiation and cell cycle arrest. This Example shows that ENH is a cytoplasmic retention factor for Id2, and that the ectopic expression of ENH in neuroblastoma cells leads to translocation of Id2 from the nucleus to the cytoplasm, with consequent inactivation of transcriptional and cell-cycle-promoting functions of Id2. Conversely, silencing of ENH by RNA interference prevents cytoplasmic relocation of Id2 in neuroblastoma cells differentiated with RA. Id2 in neuroblastoma cells differentiated with RA. The differentiated neural crest-derived tumor ganglionueroblastoma coexpresses Id2 and ENH in the cytoplasm of ganglionic cells. These data at least indicate that ENH contributes to differentiation of the nervous system through cytoplasmic sequestration of Id2. The data also suggests that ENH is a restraining factor of the oncogenic activity of Id proteins in neural tumors.

Materials and Methods:

Yeast Two-Hybrid Screening: The Proquest system (Invitrogen Life Technologies, Carlsbad, Calif.) was used for yeast two-hybrid screening. The entire coding sequence of human Id2 was subcloned into the bait plasmid pDBLeu. A human fetal brain cDNA library in pPC86 was transformed into MaV203 yeast cells and screened for interactors with the bait plasmid according to the manufacturer's protocol.

Cell Culture, Colony-Forming Assay, and Transfection: Neuroblastoma cell lines SK-N-SH, IMR-32, and LAN-1, the glioma cell lines SF188, and COS-1 cells were maintained in 10% FBS (Sigma) in DMEM (Cambrex). For colony-forming assay cells were transfected with pcDNA3-ENH or vector control. Cells were selected in G418 for 14 days, and colonies were scored in triplicate cultures. Cells were transfected by using Lipofectamine 2000 according to the manufacturer's instructions.

Northern Blot: RNA was isolated by the TRIzol (Invitrogen) method. Twenty micrograms of total RNA was electrophoresed on an agarose-formaldehyde gel and transferred to nylon membrane (Nytran SPC; Schleicher & Schuell). cDNA of human ENH was used as a probe.

GST Pull-Down Assay, Western Blot, and Coimmunoprecipitation: GST fusion proteins were purified from BL21 Star (Invitrogen). GST pull-down assay was performed as described in lavarone, A. et al., (1994), Genes Dev., 8:1270-1284. For Wetern blot analysis, cellular pellets were lysed in ice-cold RIPA buffer (50 mM Tris, pH 7.5/150 mM NaCl/1% Nonidet P-40/0.5% sodium deoxycholate/0.1% SDS) containing Complete Mini protease inhibitor pellet (Roche) and 1 mM PMSF. Lysates were electrophoresed on SDS/PAGE gels and transferred to nitrocellulose membrane (Amersham Pharmacia Biotech). Membranes were stained with antibodies against ENH, Id2 (Santa Cruz Biotechnology), and α-tubulin (Sigma), and blots were developed by using ECLWestem Blotting Detection System (Amersham Pharmacia Biotech). The anti-ENH antibody is a rabbit polyclonal that was produced in collaboration with Zymed against a peptide that is fully conserved in the mouse sequence (KQQNGPPRKHI (SEQ ID NO:1)). Coimmunoprecipitation of Id2 and ENH from cells transfected with pcDNA.3-Id2 and p3XFlag-ENH was performed as described (lavarone, A. et al., (2004), Nature, 432:1040-1045).

Luciferase Assay: The luciferase reporter construct 5xE-box-luciferase (Lasorella, A. et al., (1996) Mol. Cell. Biol., 16:2570-2578) was cotransfected with pcDNA.3-E47 and pcDNA.3 vector or pcDNA.3-Id2 and pcDNA.3-ENH into SK-N-SH cells. pCMV-β-gal was cotransfected for normalization. Twenty-four hours later luciferase and β-galactosidase activities were measured as described (Lasorella, A. et al., (2000) Nature, 407:592-598).

BrdU Incorporation Study: SK-N-SH cells were plated in Lab-Tek Chamber Slides (Nalge Nunc Intl.). Cells were transfected with plasmids expressing the empty vector, Id2, ENH, or both and an EGFP expression vector to identify transfected cells. After 24 h, cells were labeled with 10 μM BrdU for 6 h and 14 h, fixed, and stained with anti-BrdU antibody (Roche) for 1 h at room temperature. Secondary antibody was donkey anti-mouse, Cy3-conjugated (Jackson hnmunoResearch). Nuclei were counterstained with DAPI. Cells were examined on an Olympus epifluorescence microscope. BrdU-positive cells were scored by counting at least 500 GFP-positive cells in three independent experiments.

Quantitative Analysis of Subcellular Localization of Id2 and siRNA Experiments: SK-N-SH-Flag-Id2 cells untreated or treated with RA for 48 h were fixed in 4% paraformaldehyde. Flag-Id2 and endogenous ENH were immunostained by using Flag-M2 (Sigma) and ENH antibodies, respectively. SK-N-SH cells were transfected with vector or p3XFlag-ENH and immunostained by using Flag-M2 and Id2 (Zymed) antibodies. For silencing of ENH, ENH siRNA (siGenome Smartpool reagent M-006930-00) and control, nontargeting (siGenome Smartpool reagent D-001206-13) siRNA mixtures were purchased from Dharmacon. SK-N-SH cells stably expressing Flag-Id2 were treated with vehicle control or RA for 48 h and transfected with 60 nM siRNAs by using Lipofectamine 2000 (Invitrogen). Thirty-six hours after transfection cells were fixed in 4% paraformaldehyde and immunostained by using Flag-M2 antibodies. Parallel cultures were analyzed by Western blot. Secondary antibodies were FITC- or Cy3-conjugated anti-rabbit and Cy3-conjugated anti-mouse (Jackson ImmunoResearch). Nuclei were counterstained with DAPI. Slides were mounted in 90% glycerol in PBS and analyzed on an Olympus epifluorescence microscope. The percentage of cells displaying Id2 staining in the nucleus was scored by counting at least 500 cells from triplicate samples.

Transgene Construction, Generation, and Screening of Mice: To direct transgenic expression of Id2, Flag-tagged Id2 cDNA was driven by the enhancer element contained in the second intron of the Nestin gene coupled with the thymidine kinase minimal promoter (Chenn, A. and Walsh, C. A., (2002), Science, 297:365-369). The second intron of the Nestin gene directs expression in central nervous system progenitor cells. The first intron from the rat insulin II gene was included to enhance expression levels (Yaworsky, P. J. and Kappen, C. (1999), Dev. Biol., 205:309-321). The transgene fragment was microinjected at a concentration of 6 ng/μl into fertilized mouse eggs. Transgenic mice were identified by PCR analysis of DNA samples prepared from tail biopsies.

Immunohistochemistry. GNB sections were from anonymous tumors stored in the Columbia University tumor bank. Sections from E15.5 mouse brain or primary tumors were deparaffinized in xylenes and rehydrated in a graded series of ethyl alcohol. Primary antibodies were Flag-M2 (Sigma), Id2, and ENH(Zymed). Avidin-biotin-peroxidase complex technique was used for primary antibody detection (Vectastain kit; Vector Laboratories). Staining was developed by using diaminobenzidine (brown precipitate). Sections were counterstained with hematoxylin. Rabbit or mouse IgG (Vector Laboratories) and tissue from Id2^{-/-} mice were routinely used as controls for specificity of the staining.

The LIM Domains of ENH Bind to the Helix-Loop-Helix (HLH) Domain of Id Proteins:

To identify new interactors of Id2 from the nervous system, a yeast two-hybrid screen was conducted with a human fetal brain cDNA library using full length Id2 as bait. This screen yielded 47 validated cDNA clones corresponding to four different Id2-associated proteins. Among them, 24 clones code for Id2, 13 clones code for the bHLH transcription factor E2-2, eight clones code for the bHLH transcription factor HEB, and two clones code for the PDZ-LIM protein ENH. All Id2 and bHLH clones retain an intact HLH domain. This finding is consistent with the essential role of the HLH domain for heterodimerization. The presence of endogenous Id2 is explained by the strong homodimerization ability of Id2 and its abundant expression in the fetal brain. The identification of two E proteins, E2-2 and HEB, demonstrated that the screen was capable of identifying specific Id2 interactors.

The only two clones that did not contain a HLH domain code for ENH, a member of the Enigma family of LIM domain proteins, a class of proteins associated with the actin cytoskeleton (Khurana, T. et al., (2002), Protoplasma, 219:1-12; Nakagawa, N. et al., (2000), Biochem. Biophys. Res. Commun., 272:505-512; Vallenius, T. et al., (2000) J. Biol. Chem., 275:11100-11105; Ueki, N. et al., (1999) J. Hum. Genet., 44:256-260).

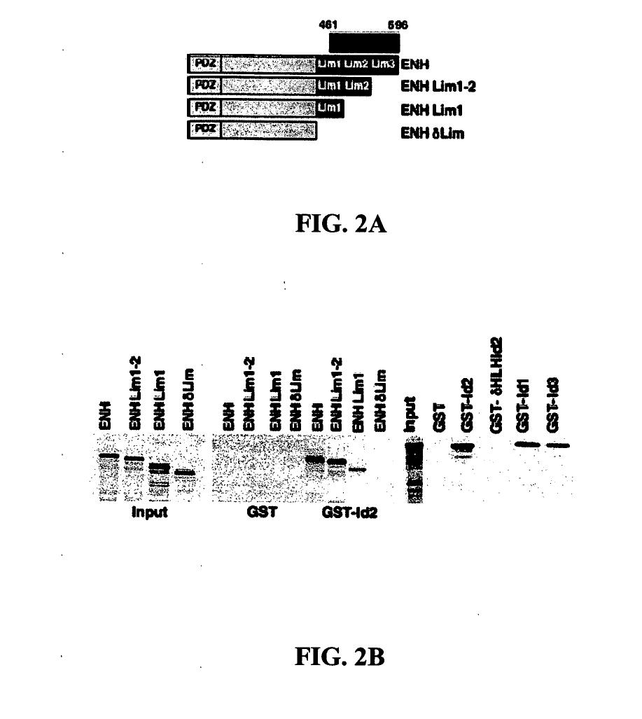

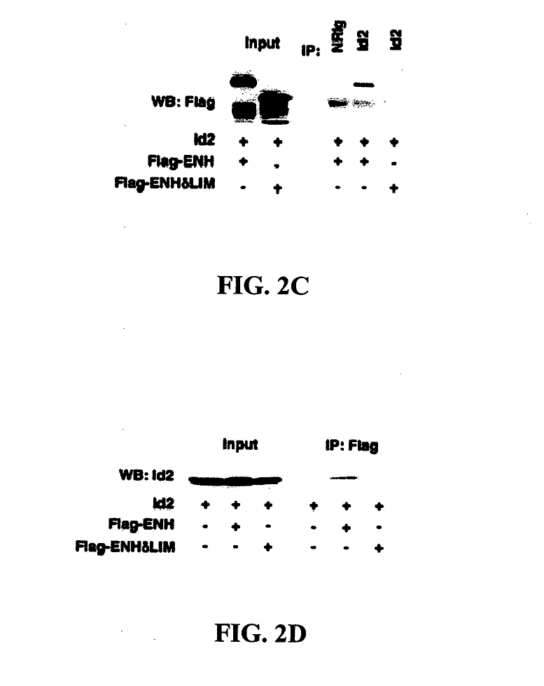

Proteins of the Enigma family possess an N-terminal PDZ domain and three LIM domains at the C terminus (FIG. 2A). All members of the PDZ-LIM Enigma family, including ENH, are cytoplasmic proteins that bind to the actin cytoskeleton through direct interaction between the PDZ domain and α-actinin (Nakagawa (2000); Vallenius (2000)). Sequence analysis of the two ENH clones identified in the two-hybrid assay established that both clones retained a C-terminal fragment of ENH (amino acids 461-596, see SEQ ID NO:4) that includes part of the first and the last two LIM domains but lacked the N-terminal region with the PDZ domain (FIG. 2A).

To validate the specificity of the binding between ENH and Id2 and identify the domains that mediate this interaction, GST fusion proteins and in vitro-translated proteins in pull-down assays.

GST-Id2 bound efficiently to in vitro-translated, 35S-labeled full-length ENH and to C-terminal ENH deletion constructs that retain two (ENH LIM1-2) or one (ENH LIM1) LIM domains. However, GST-Id2 did not bind to an ENH polypeptide lacking all LIM domains (ENHδLIM) (FIG. 2B Left and Center). GST-Id2 fusion protein carrying a deletion of the HLH domain (GST-δHLHId2) failed to bind ENH (FIG. 2BRight).

Given the high homology among the HLH domains of the three Id proteins, it was asked whether Id1 and Id3 could also bind ENH in this assay. Indeed, both GST-Id1 and GST-Id3 bound to full-length ENH (FIG. 2B Right).

To determine whether Id2 binds ENH in vivo, coimmunoprecipitation experiments were performed after transfecting Id2 and Flag-tagged ENH in Cos-1. Anti-Id2 antibodies precipitated full-length Flag-ENH but not Flag-ENHδLIM (FIG. 2C). Accordingly, Flag-ENH but not Flag-ENHδLIM immunoprecipitates contained Id2 (FIG. 2D). Together, these results confirmed that binding of Id2 to ENH occurred through a specific interaction between the LIM domains of ENH and the HLH domain of Id2.

ENH is Expressed in the Nervous System and is Induced During Differentiation:

The role of ENH has been mostly studied in cardiac and skeletal muscle cells, where ENH has been proposed as a key factor for the integrity of the actin cytoskeleton in differentiated myocytes. However, recent reports show that ENH binds to the N-type calcium channel and suggest that a PKC-ENH-calcium channel complex regulates channel activity in neurons.

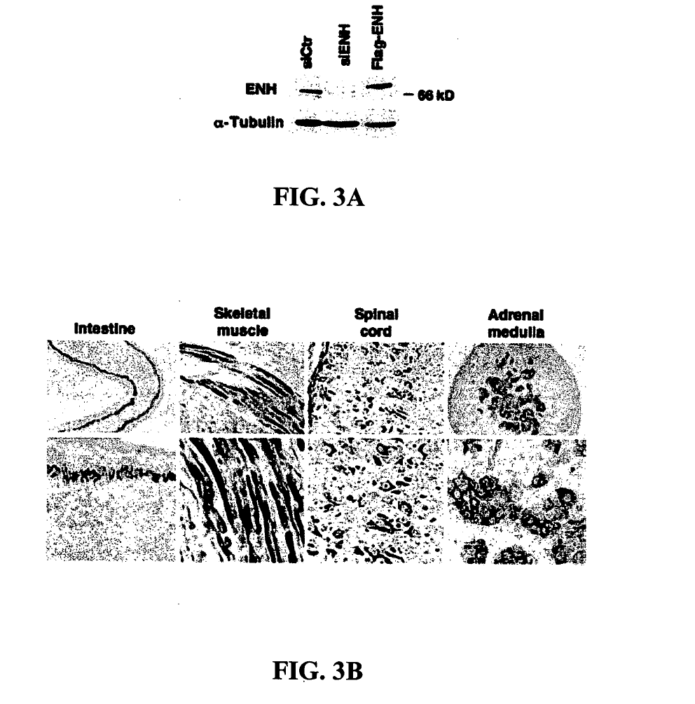

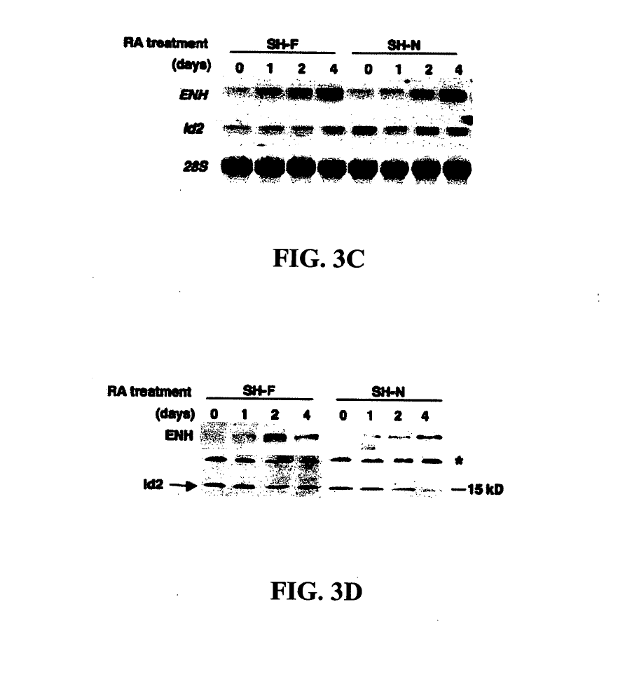

Herein, isolation of ENH from a fetal brain cDNA library suggests that this protein may be implicated in neural development as well. To conduct this study, a polyclonal antibody was raised against a peptide (SEQ ID NO:1) shared by human and mouse ENH. From Western blot experiments, it was confirmed that the antibody interacts specifically with exogenously expressed Flag-ENH and endogenous ENH from RA-treated SK-N-SH neuroblastoma cells (FIG. 3A).

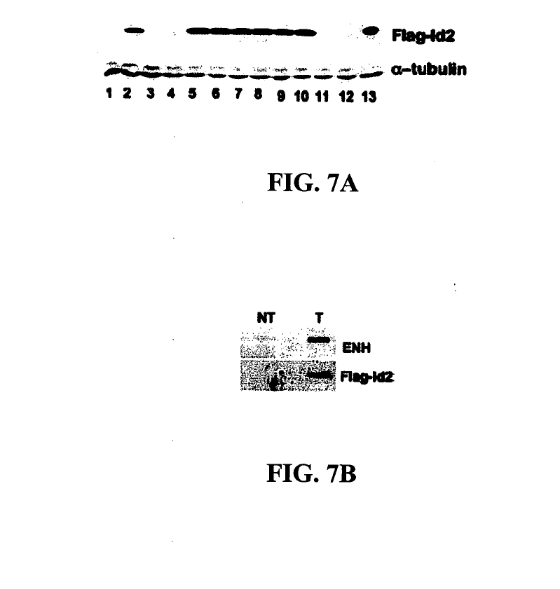

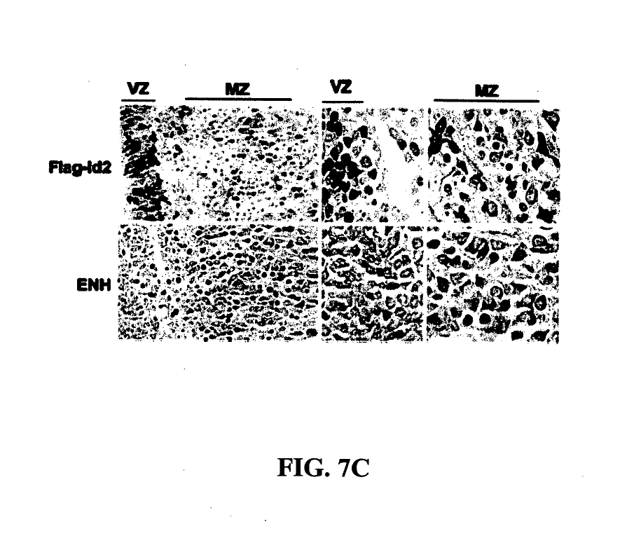

To test whether ENH is expressed during mouse development, sections were stained from embryonic day 15.5 (E15.5) mouse embryo with the anti-ENH antibody. In agreement with published observations, smooth and skeletal muscle cells were strongly stained. At this developmental age, ENH is clearly detectable in neurons from the central nervous system (see the positive ENH staining in spinal cord neurons in FIG. 3B) and dorsal root ganglia as well as in chromaffin cells of the adrenal medulla, thus suggesting that expression of ENH is more widespread than it has been previously reported (FIG. 3B; see also FIG. 7C and data not shown for dorsal root ganglia).

Human neuroblastoma cells are frequently used as in vitro models to recapitulate differentiation of the nervous system. To ask whether ENH expression is regulated during differentiation of the nervous system, clonal derivatives of the human neuroblastoma cell line SK-N-SH, the SK-N-SH-N (SH-N) and SK-N-SH-F (SH-F) cells were used. These cells, which lack N-myc gene amplification, have been used to characterize the cell-cycle exit associated with differentiation of neural cells. When treated with a low concentration of RA (0.1 μM) SH-N cells undergo differentiation along the neuronal lineage, whereas SH-F cells acquire an epithelioid morphology and rapidly enter into a senescent-like state. Both cell types arrest in the G1 phase of the cell cycle within 48 h of treatment with RA. Remarkably, RA induced progressive elevation of ENH mRNA and protein in SH-N and SH-F cells, suggesting that ENH may play a role in multiple differentiation pathways in the nervous system (FIG. 3B and C). Although higher concentrations of RA led to marked inhibition of N-myc and Id2 gene expression in N-myc-amplified neuroblastoma cells, it was noted that RA at the concentration of 0.1 μM caused little change of Id2 expression in the SK-N-SH derivatives. However, a late decrease of Id2 protein was evident in RA-treated SH-N cells (FIG. 3D).

ENH is Essential for Cytoplasmic Relocation of Id2 in Neuroblastoma Cells Treated with RA:

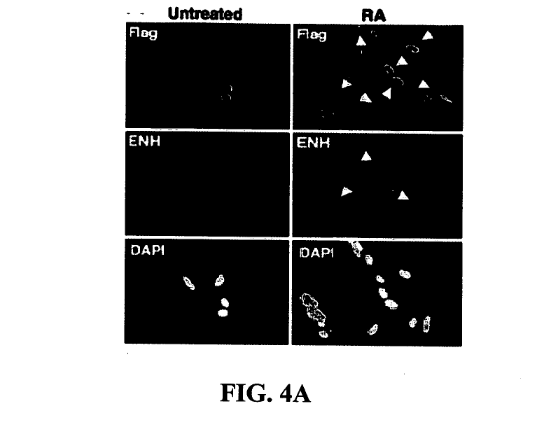

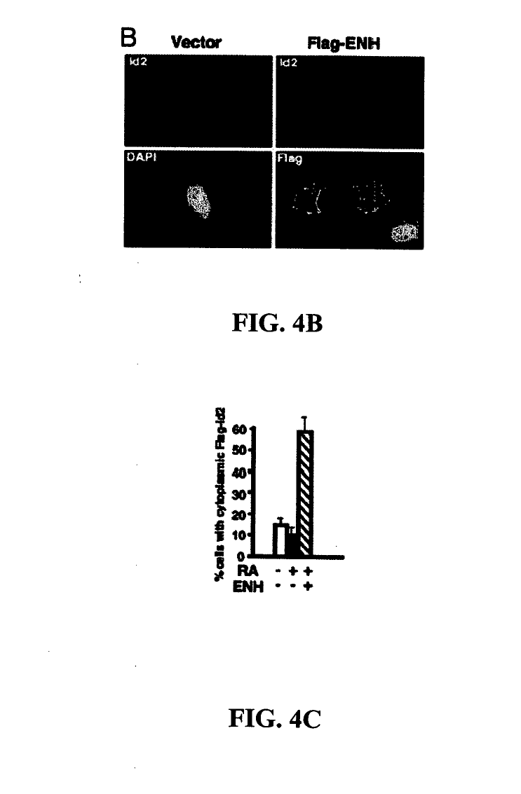

It was asked whether elevation of ENH in RA-treated neuroblastoma cells leads to sequestration of Id2 in the cytoplasm by two independent experimental approaches. First, the subcellular localization of Flag-Id2 was examined after treatment with RA of SK-N-SH using double immunofluorescence staining of endogenous ENH and Flag. Flag-Id2 was predominantly nuclear in untreated cells (FIG. 4A Top Left). In agreement with results shown in FIG. 3, logarithmically growing neuroblastoma cells showed minimal ENH staining (FIG. 4A Middle Left). After treatment with RA, Flag-Id2 relocated to the cytoplasm in cells that had acquired high ENH expression (FIG. 4A Top Right, arrows; see also Middle for expression of ENH in the same cells) but remained nuclear in ENH-negative cells (FIG. 4A Top Right, arrowheads). Quantitative analysis of the subcellular localization of Id2 and ENH from three independent experiments demonstrated that, after treatment with RA, Flag-Id2 relocated to the cytoplasm in ˜60% of the ENH positive cells compared with 10% of the ENH-negative cells and 15% of untreated cells (FIG. 4C).

Next ectopic Flag-ENH was introduced in SK-N-SH and the subcellular localization of endogenous Id2 was examined. Ectopic ENH localized to the cytoplasm with a pattern compatible with actin stress fibers (FIG. 4B Lower Right). As expected, Id2 was mainly nuclear in cells transfected with empty vector (FIG. 4B Left). However, expression of ENH caused translocation of Id2 to the cytoplasm (FIG. 4B Upper Right).

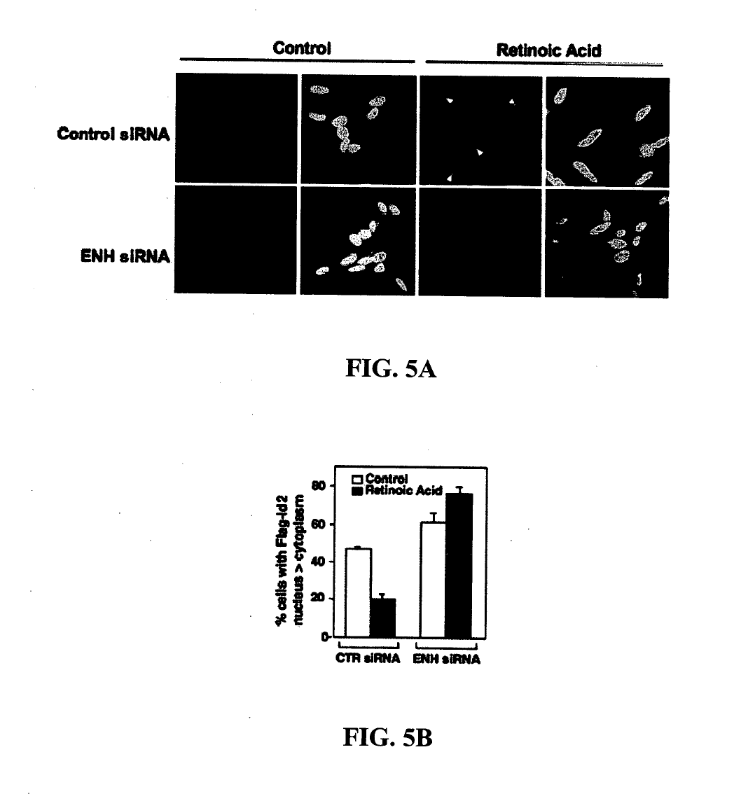

To establish the functional significance of endogenous ENH for the cytoplasmic relocation of Id2 induced by RA, a loss-of-function approach using small interfering RNA (siRNA) oligonucleotides directed to ENH was used. Transfection of RA-treated SK-N-SH with siRNA targeting ENH resulted in efficient depletion of ENH from these cells (FIG. 3A). Flag-Id2 translocated to the cytoplasm of RA-treated SK-N-SH in the presence of scrambled siRNA oligonucleotides, but the siRNAmediated silencing of ENH prevented entirely the RA-induced relocation of Flag-Id2 to the cytoplasm (FIG. 5A; see also FIG. 5B for the quantitative analysis of subcellular localization of Flag-Id2). Together, these results indicate that activation of ENH expression by RA is essential for cytoplasmic sequestration of Id2 in neuroblastoma.

Thus, similar binding experiments can be conducted to determine whether other PDZ-LIM family proteins (including domains, mutants, or variants thereof) can specifically bind to an Id protein. Further, similar immunofluorescence-based subcellular localization experiments can be conducted to determine whether other PDZ-LIM family proteins (including domains, mutants, or variants thereof) can cause an Id protein to translocate to, relocate to, be retained in, or be sequestered in, the cytoplasm of a cell. Further, the above subcellular localization experiments can be conducted with essentially any cell, including primary cells, cells obtained from tissues, and cells from or cell-lines established from tumors/cancers, including small cell carcinoma (SCC) of the head, neck, esophagus and oral cavity; melanoma; hepatocellular carcionoma; colorectal adenocarcinoma; pancreatic cancer; medullary thyroid cancer; papillary thryoid cancer; astrocytic tumor; neuroblastoma; Ewing's sarcoma; ovarian tumor; cervical cancer; endometrial carcinoma; breast cancer; prostate cancer; and malignant seminoma.

ENH Counters Id2 Activity and is an Inhibitor of Proliferation and Cell-Cycle Progression:

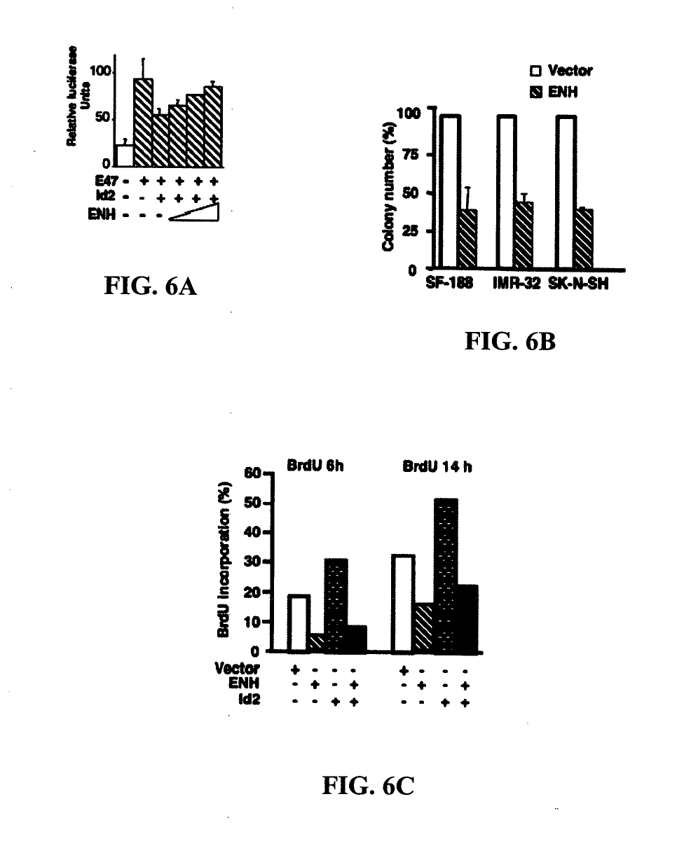

To test the hypothesis that ENH restrains the inhibitory effects of Id2 on bHLH-mediated transcription by acting as a cytoplasmic retention factor for Id2, luciferase reporter assays were performed with five multimerized E-boxes driving expression of luciferase (E-box-luc). The E-box-luc plasmid was transfected in the presence of mammalian expression vectors for the ubiquitously expressed bHLH protein E47, Id2, and increasing amounts of ENH. Id2 inhibition of E47-mediated transcription was relieved by coexpression of ENH in a dose-dependent manner (FIG. 6A).

A well known function of Id2 is the ability to enhance cell proliferation by promoting the transition from G1 to S phase of the cell cycle. Therefore, it was asked whether ENH inhibited cell proliferation and opposed Id2-mediated entry into S phase. Expression of ENH in three human neuroectodermal cell lines (the glioma cell line SF188 and the neuroblastoma cell lines IMR-32 and SK-N-SH) markedly inhibited colony formation, suggesting that ENH has antiproliferative effects (FIG. 6B). Next SK-N-SH was transfected with ENH and Id2 in the presence of a GFP expression plasmid and measured the rate of DNA synthesis by incorporation of BrdU of the successfully transfected, GFP-positive cells. Ectopic ENH strongly inhibited S phase entry and abrogated the Id2-mediated stimulation of DNA synthesis (FIG. 6C). These results suggest that, through its ability to sequester Id2 in the cytoplasm, ENH can efficiently suppress the functions of Id2 requiring nuclear localization, including the stimulation of cell-cycle progression.

In a similar manner, any agent contemplated by the invention can be tested to determine whether the agent can inhibit Id mediated cell proliferation. Briefly, cells can be transfected with an expression vector encoding an Id protein(s) and either incubated with a chemical compound agent or transfected with an expression vector encoding a protein agent, such as a protein or peptide comprising a LIM domain and/or a PDZ domain. The cells can also be transfected with a plasmid encoding GFP to identify transfected cells, because the principle of co-transfection provides that multiple plasmids can be transfected into cells. After transfection, cell cultures are labeled with BrdU and immunostained for BrdU using a primary antibody specific to BrdU and a secondary antibody conjugated with a fluorophore, such as Cy3. Cells are then assessed for GFP and BrdU, and the percentage of transfected cells positive for BrdU are scored. When the percentage of transfected cell positive for BrdU is reduced as compared to control experiments (where an agent is not added or transfected into the cells), this indicates that the agent can inhibit Id mediated cellular proliferation.

Further, the luciferase reporter assays can also be used to test whether other proteins of the invention can restrain the inhibitory effect of Id2 on bHLH-mediated transcription by acting as a cytoplasmic retention factor for Id2.

The ENH-Id2 Pathway in Development and Cancer from the Nervous System:

Taken together, the above findings indicate that, even when ectopically expressed, Id2 may be efficiently inactivated through cytoplasmic relocation implemented by differentiation signals that converge upon up-regulation of ENH. To test this hypothesis in a genetic mouse model in vivo, transgenic mice were generated expressing Flag-Id2 from the neural-specific promoter Nestin. Six independent Nestin-Flag-Id2 mouse transgenic lines were established.

It was confirmed that Flag-Id2 is expressed in the telencephalon of hemizygous embryos by Western blot (FIG. 7A) and immunohistochemistry (FIG. 7C Upper). The older transgenic mice of this colony are >1 year old. No abnormality in growth and differentiation of the nervous system during embryogenesis or postnatal life of Nestin-Flag-Id2 transgenics was observed. Thus, this transgenic system was used to ask whether normal differentiation in the nervous system requires ENH-mediated relocation of Id2 to the cytoplasm. (This model can be similarly used with other Id proteins, i.e., Nestin-Flag-Id1, Id3, or Id4 transgenic mice).

First, coimmunoprecipitation experiments were used to show that Flag-Id2 interacted specifically with endogenous ENH in Nestin-Flag-Id2 transgenic brains (FIG. 7B). Next, immunohistochemistry was performed for Flag and ENH on adjacent sections of the telencephalon at E15.5. At this developmental age, active proliferation of neural precursors is present in the periventricular, germinal layer [ventricular zone (VZ)], whereas differentiated neurons migrate radially and enter the mantle zone (MZ), which contains postmitotic cells. Flag-Id2 was predominantly nuclear in the neural precursors of the VZ but relocated to the cytoplasm in the differentiating neurons migrating toward the MZ (FIG. 7C Upper). Interestingly, ENH was barely detectable in the proliferating and undifferentiated precursors of the VZ but was coexpressed with Id2 in the cytoplasm of differentiated neurons (FIG. 7C Lower). These findings suggest that ENH is a component of the physiologic neural differentiation machinery that promotes cytoplasm relocation of Id2 in the developing brain.

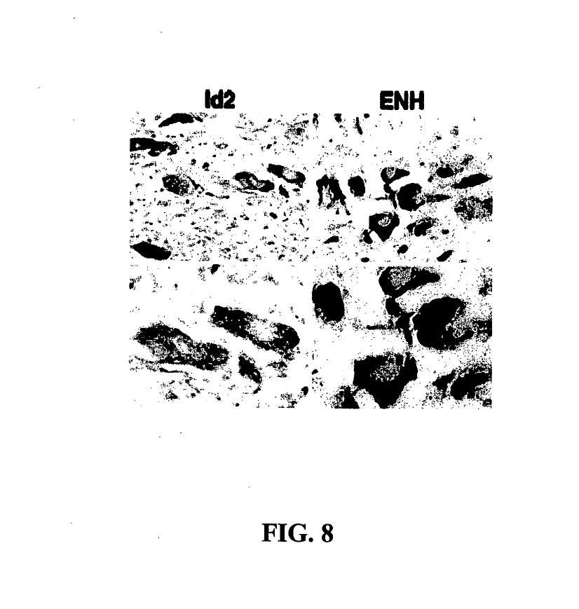

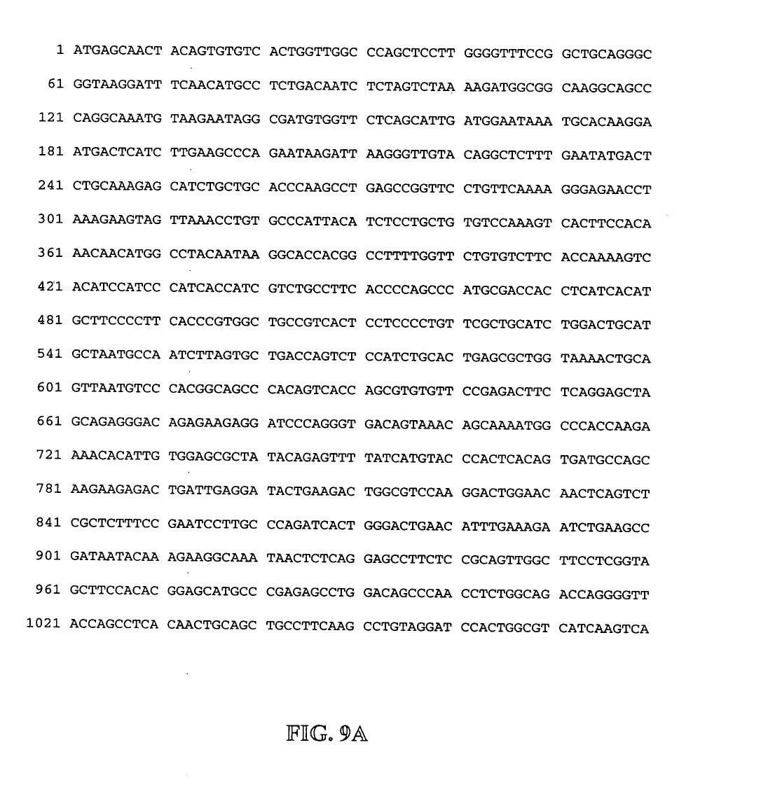

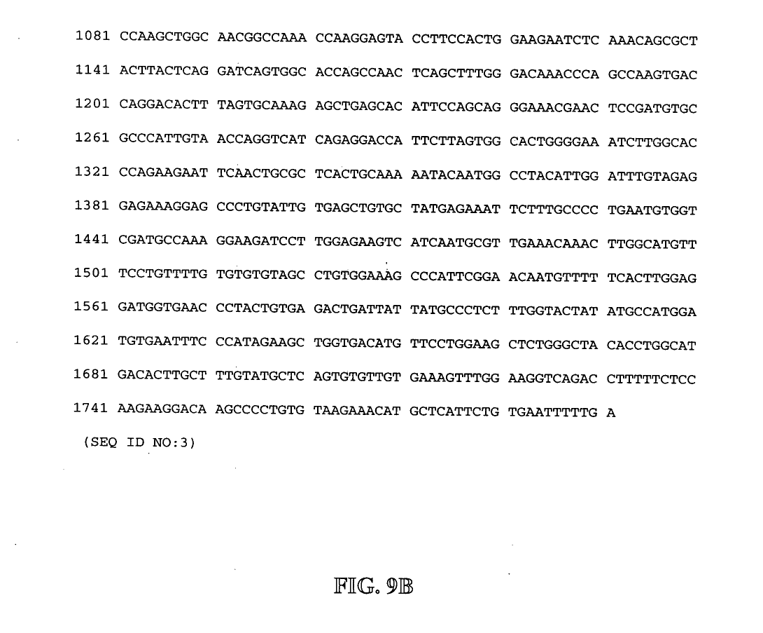

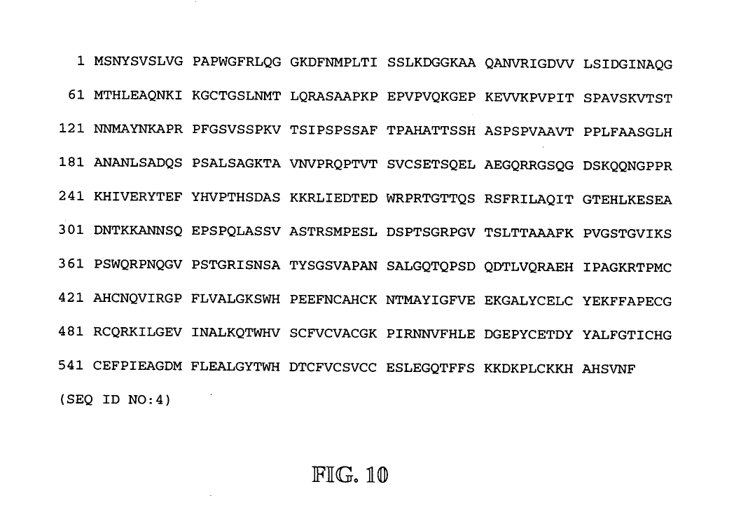

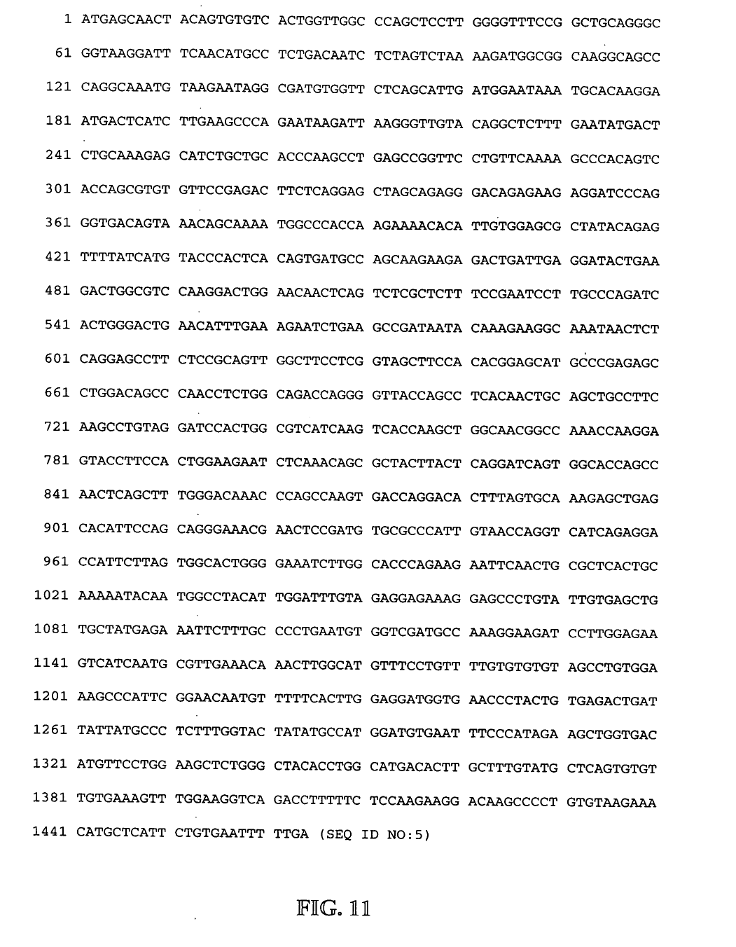

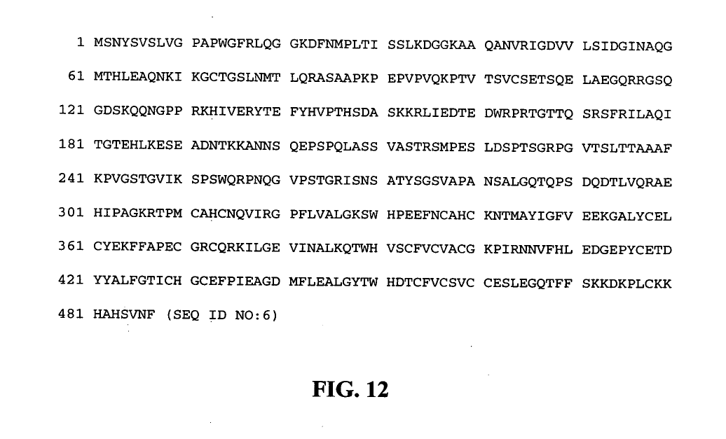

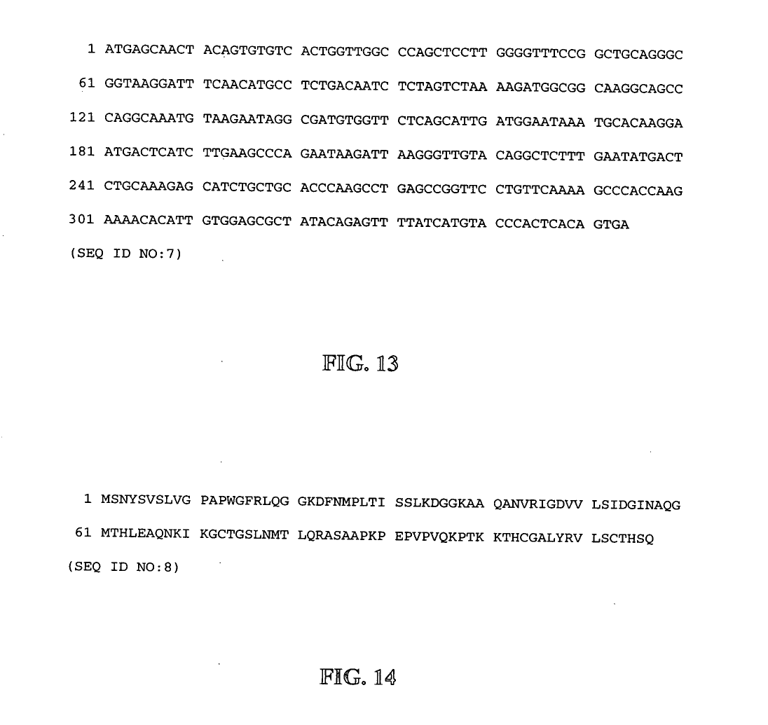

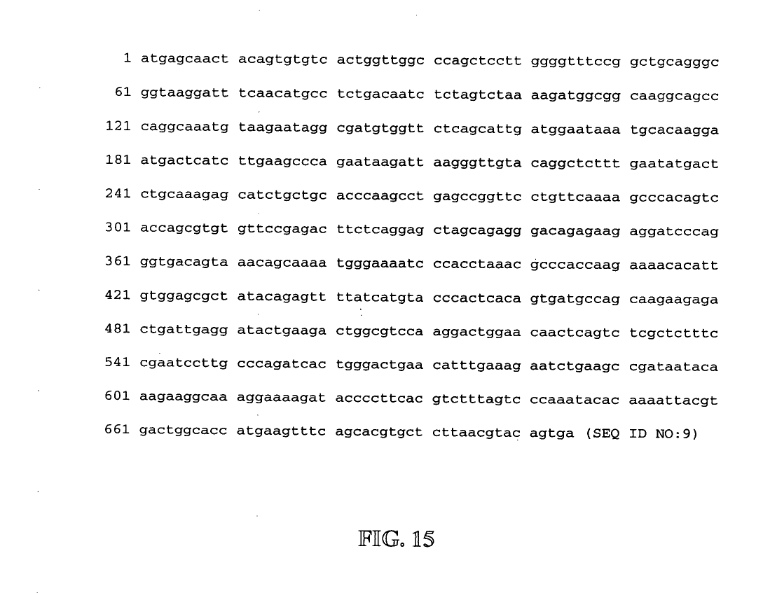

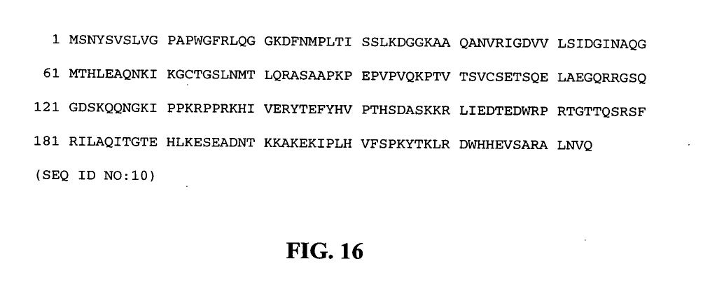

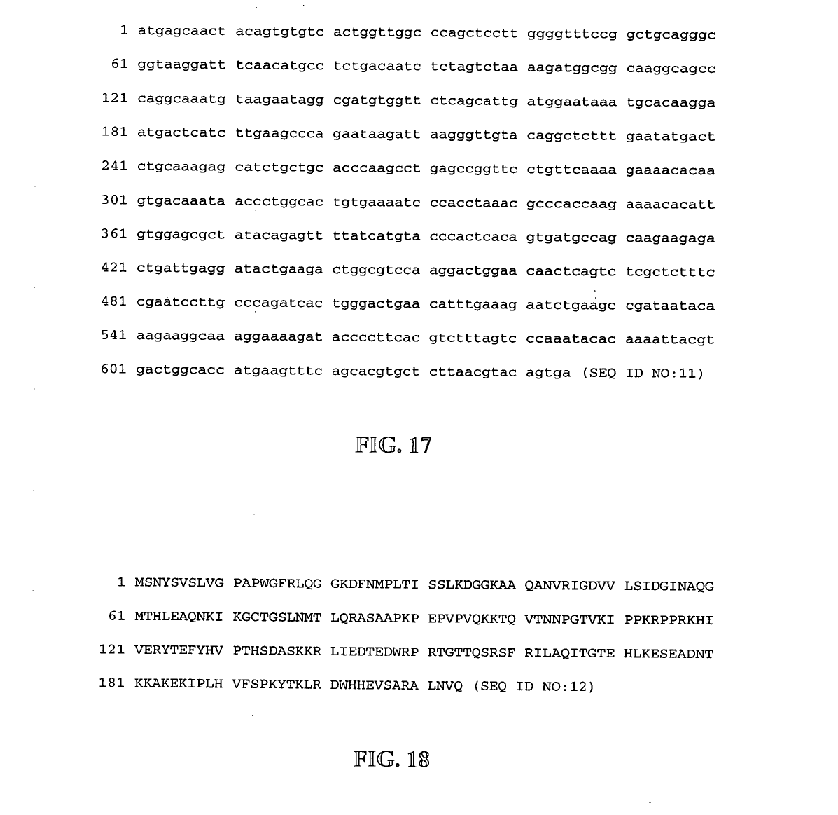

It was established that Id2 displays predominant nuclear expression in aggressive neuroblastoma, an undifferentiated form of pediatric tumor derived from the neural crest. A more differentiated form of these tumors, the ganglioneuroblastoma (GNB), is characterized by the presence of a differentiated cellular component interspersed within a predominant, undifferentiated population of cells. To determine whether ENH may regulate the subcellular compartmentalization of Id2 in primary neural tumors, the expression of ENH and Id2 was compared in four GNBs by immunostaining tumor sections with antibodies against Id2 and ENH. Most of the tumor cells stained negative for both ENH and Id2. However, cytoplasmic accumulation of Id2 was detected in the mature ganglionic cells of these tumors. Interestingly, the cytoplasm of the same cells was markedly positive for ENH. Representative images are shown in FIG. 8. The tumor expression data support the above findings in cell culture and embryonic mouse brain, and further strengthen the hypothesis that differentiation of neural cells requires ENH to sequester Id2 in the cytoplasm. This indicates that ENH may play a role in reducing the malignant behavior of neuroectodermal tumors through the sequestration of Id2 in the cytoplasm.

Additional Assays

To test agents of the invention for their ability to treat tumors in vivo, tumors can be explanted into nude mice (i.e., athymic mice). After the tumors are established in the mice, the agents contemplated by the invention can be administered to the mice in order to test for whether the agents can diminish the tumors.

Further, to determine whether an agent contemplated by the invention can cause tumor cells to become more differentiated and less malignant in vivo, the SH-N-Flag-Id2 cell line (or a SH-N-Flag-Id1 (or -Id3, or -Id4) cell line) that expresses aberrant amounts of primarily nuclear Id can be used. Other tumor cell lines that stably express an Id protein can also be used. For this purpose, vectors that carry the puromycin resistance gene and express a “LIM/PDZ protein” comprising: (1) at least one LIM domain, (2) at least one PDZ domain, or (3) at least one LIM domain and at least one PDZ domain can be generated. They can be used to produce SH-N-Flag-Id stable cell lines (or other tumor cell lines stably expressing an Id protein) co-expressing Id and the LIM/PDZ protein.

The SH-N-Flag-Id cell lines that do or do not express an LIM/PDZ protein can be implanted into the kidney capsule of nude mice (Huang, J. et al., Proc. Natl. Acad. Sci. USA, 100:7785-90; Kim, E. et al., Proc. Natl. Acad. Sci. USA, 99:11399-404.) This mouse model has been used by the invention to demonstrate a key role for Id2 in promoting proliferation and neoangiogenesis in SH-N neuroblastoma cells through the enhanced production of VEGF. Thus, if a LIM/PDZ protein can sequester Id in the cytoplasm, then it is expected that the LIM/PDZ protein can oppose some or all Id-mediated activities that stimulate tumor growth, including neoangiogenesis. Additionally, SH-N-Flag-Id cell lines (or other tumor cell lines that express an Id protein) can be used to generate mouse tumor models, such that chemical compounds can be administered to these mouse models to assess whether the compounds can oppose Id-mediated tumor growth.

To determine whether an agent can oppose Id-mediated tumor growth, tumor size can be measured in the absence or presence of an agent. Further, the proliferative index of SH-N-Flag-Id tumors (or other Id-comprising tumor cell line tumors) can be determined in the absence or presence of an agent by quantitating cells positive for BrdU (S phase) and phospho-histone H3 (M phase). Through immunostaining for neuronal and glial markers, the differentiation stage of neural tumors can also be assessed, i.e., to determine whether an agent can promote the differentiation of a tumor such that it becomes less malignant. To assess whether an agent inhibits angiogenesis, the presence of an agent should result in impaired expression of VEGF with disrupted vascularity in the tumors. Tumor angiogenesis in mouse tumor models can be examined at a macroscopic (through injectin of Orange microfil latex) and at a microscopic level (expression of markers for new tumor blood vessels such as PECAM and SMA). The expression of VEGF can be scored by immunohistochemistry and western blot analysis of the different tumors.

Discussion of Experimental Results:

Id2 activity is primarily executed in the nucleus, where the Id2 protein antagonizes the function of DNA binding proteins and pocket proteins of the Rb family. Although other biological conditions may regulate subcellular compartmentalization of Id2, the process of differentiation, associated with the state of proliferative quiescence, requires nuclear exclusion of Id2. Here, it has been determined that the cytoskeleton-associated protein ENH binds and sequesters Id2 in the cytoplasm, thus preventing its nuclear actions.

ENH belongs to a growing family of adaptor proteins that are anchored to the actin cytoskeleton through the PDZ domain and direct LIM-associated partners to actin filaments. The LIM domains of ENH are cysteine-rich double zinc finger motifs, which are known to mediate protein-protein interactions. They contact the HLH domain of Id2. The findings herein suggest that the LIM-HLH interaction is used by ENH to inhibit nuclear shuttling of Id2 and drive differentiation. Knockdown of ENH had marked consequences on the cytoplasmic translocation of Id2 promoted by treatment of neuroblastoma cells with RA, a powerful inhibitor of cell proliferation and inducer of multiple pathways of differentiation. By anchoring itself to the actin cytoskeleton through the N-terminal PDZ domain, ENH can tether Id2 to the cytoskeleton. This mechanism recapitulates that ascribed to other cytoskeleton-associated proteins for their ability to sequester transcription factors in the cytoplasm.

Although the experiments in this Example have been focused primarily on the functional interaction between ENH and Id2 in neural cells, the ability of ENH to bind other Id proteins combined with its participation in differentiation of other tissue types (e.g., muscle) suggests that ENH may be a general inducer of differentiation through binding and cytoplasmic sequestration of Id proteins.

The ENH isoforms c, d, and e lack the three LIM domains and resemble the ENHδLIM mutant tested by in FIG. 2A and B. Based on the results, it can be concluded that the alternative ENH isoforms lacking LIM domains are unable to bind Id2 (or other Id proteins), a property that might contribute to a potential dominant-negative activity toward full-length ENH (ENH1 or SEQ ID NO:4) in vivo.

Id proteins are aberrantly accumulated in various forms of human cancer, where they drive multiple hallmarks of neoplasia. The most common mechanism selected by tumor cells to activate Id function is to elevate the expression of Id genes through oncogenic activation of the upstream transcriptional enhancers. The data herein suggest that tumor cells may also target another level of regulation of the Id biology. The findings in GNB implicate that, by limiting the access of Id2 to the nuclear targets, expression of ENH may be a crucial safeguard against full-blown anaplasia in more differentiated tumors.

Like ENH, other members of the PDZ-LIM domain family of proteins may be more abundantly expressed in non-transformed cells and suppress growth of tumor cells. It is possible that this is a general attribute of this family of proteins. Thus, the invention provides for the testing of whether the most aggressive forms of human neoplasm select genetic and/or epigenetic alterations of the genes coding for PDZ-LIM proteins. Further, the invention provides for methods of treating these neoplasias by introducing LIM comprising proteins that can sequester and inhibit Id protein function.

What is claimed:

1. A method for inhibiting proliferation of a neoplastic cell or for promoting differentiation of the neoplastic cell, the method comprising expressing in the neoplastic cell a protein comprising at least one LIM domain(s), wherein the protein sequesters an Id protein in the cytoplasm of the cell, thereby inhibiting proliferation.

2. A method for inhibiting angiogenesis in a tumor, the method comprising expressing in a cell of the tumor a protein comprising at least one LIM domain, wherein the protein sequesters an Id protein in the cytoplasm of the cell, thereby inhibiting angiogenesis.

3. A method for treating cancer in a subject, the method comprising administering to the subject an effective amount of a protein comprising at least one LIM domain, or an effective amount of a nucleic acid comprising a coding sequence for the protein, wherein the protein sequesters an Id protein in the cytoplasm of a cancer cell in the subject, thereby treating cancer in the subject.

4. The method of claim 1, 2, or 3, wherein the LIM domain(s) binds to the Id protein.

5. The method of claim 4, wherein the binding of the protein to the Id protein results in a protein-complex that is greater than about 40 kilodaltons, thereby preventing the Id protein from migrating into the nucleus of the cell.

6. The method of claim 1, 2, or 3, wherein the protein translocates the Id protein to the cytoplasm, thereby sequestering the Id protein in the cytoplasm.

7. A method for treating cancer in a subject, the method comprising administering to the subject an effective amount of a chemical compound, wherein the chemical compound sequesters an Id protein in the cytoplasm of a cancer cell in the subject, thereby treating cancer in the subject.

8. The method of claim 7, wherein the chemical compound is a peptidomimetic of a PDZ-LIM protein.

9. The method of claim 8, wherein the peptidomimetic mimics the structure of a LIM domain of the PDZ-LIM protein.

10. The method of claim 9, wherein the PDZ-LIM protein is ENH.

11. The method of claim 1, 2, 3, or 7, wherein the cell is from a small cell carcinoma, a melanoma, a hepatocellular carcionoma, a colorectal adenocarcinoma, a pancreatic cancer, a medullary thyroid cancer, a papillary thyroid cancer, an astrocytic tumor, a neuroblastoma, Ewing's sarcoma, an ovarian tumor, a cervical cancer, an endometrial carcinoma, a breast cancer, a prostate cancer, a nervous system tumor, Wilms' tumor, a retinoblastoma, or a malignant seminoma.

12. The method of claim 1, 2, or 3, wherein the protein further comprises at least one PDZ domain.

13. The method of claim 12, wherein the LIM domain(s) bind to the Id protein, and wherein the PDZ domain(s) binds to a cytoskeletal protein of the cell, thereby sequestering the Id protein in the cytoplasm.

14. The method of claim 1, 2, or 3, wherein the protein is a PDZ-LIM protein, or a mutant or a variant thereof.

15. The method of claim 1, 2, or 3, wherein the protein is ENH, or a mutant or a variant thereof.

16. The method of claim 3, wherein the protein is administered to the subject in a liposome.

17. The method of claim 3, wherein the nucleic acid is an expression vector.

18. The method of claim 17, wherein the expression vector is a viral vector.

19. The method of claim 17, wherein the nucleic acid comprises a recombinant genome of a virus particle, wherein the virus particle is administered to the subject.

20. The method of claim 1, 2, 3, or 7, wherein the Id protein is Id2.

21. The method of claim 1, 2, 3, or 7, wherein the cell or the cancer cell is a neuroblastoma cell.

22. A method for grading a tumor, the method comprising determining whether a PDZ-LIM protein is expressed in a tumor cell, wherein if the PDZ-LIM protein is expressed then the tumor is graded higher or as a more aggressive tumor than if the PDZ-LIM is not expressed.

23. The method of claim 22, wherein the PDZ-LIM protein is ENH.

24. A method for identifying an agent that can sequester an Id protein in the cytoplasm of a cell and/or translocate an Id protein to the cytoplasm of the cell, the method comprising:
(a) incubating the agent with the cell, wherein an Id protein is present in the nucleus of the cell prior to incubation; and
(b) determining whether the Id protein of the cell is sequestered in and/or translocated to the cytoplasm of the cell, wherein if the Id protein is determined to be sequestered in and/or translocated to the cytoplasm, then the agent is identified as an agent that can sequester an Id in the cytoplasm and/or translocate an Id protein from the nucleus to the cytoplasm of a cell.

25. The method of claim 24, wherein the cell comprises a cancer cell.

26. The method of claim 25, wherein the cancer cell comprises a cell from a small cell carcinoma, a melanoma, a hepatocellular carcionoma, a colorectal adenocarcinoma, a pancreatic cancer, a medullary thyroid cancer, a papillary thyroid cancer, an astrocytic tumor, a neuroblastoma, Ewing's sarcoma, an ovarian tumor, a cervical cancer, an endometrial carcinoma, a breast cancer, a prostate cancer, a nervous system tumor, Wilms' tumor, a retinoblastoma, or a malignant seminoma.

27. The method of claim 24, wherein the cell comprises a neuroblastoma cell.

28. The method of claim 24, wherein the Id protein is Id2.

29. The method of claim 24, wherein the determining step comprises immunofluorescence.

30. The method of claim 24, wherein the incubating step comprises incubating the agent with the cell from between about 30 minutes to about 72 hours.

31. A medical device comprising a protein comprising a LIM domain, or a chemical compound, wherein the protein or the chemical compound sequesters an Id protein in the cytoplasm of a cell and/or translocates an Id protein to the cytoplasm of a cell.

32. The medical device of claim 31, wherein the medical device is a stent, a transdermal patch, or an implantable biodegradable gel.

33. The medical device of claim 31, wherein the protein comprises ENH, or a mutant or a variant thereof.

34. The method of claim 31, wherein the Id protein is Id2.


SEQUENCE LISTING (12 sequences)

SEQ ID NO: 1 - protein, 11 aa, Homo sapiens
KQQNGPPRKHI

SEQ ID NO: 2 - protein, 45 aa, Artificial
  CONSENSUS SEQUENCE FOR A LIM DOMAIN
  MISC_FEATURE (2)..(3): X represents any amino acid
  MISC_FEATURE (5)..(19): X represents any amino acid; also this stretch can alternatively be an additional five amino acids long where the five amino acids can be any amino acid
  MISC_FEATURE (28)..(29): X represents any amino acid
  MISC_FEATURE (32)..(33): X represents any amino acid
  MISC_FEATURE (35)..(36): X represents any amino acid
  MISC_FEATURE (38)..(40): X represents any amino acid
CXXCXXXXXXXXXXXXXXXFYWHPCRHXXCHXXCXXCXXXLIVMF

SEQ ID NO: 3 - DNA, 1791 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagggagaacctaaagaagtagttaaacctgtgcccattacatctcctgctgtgtccaaagtcacttccacaaacaacatggcctacaataaggcaccacggccttttggttctgtgtcttcaccaaaagtcacatccatcccatcaccatcgtctgccttcaccccagcccatgcgaccacctcatcacatgcttccccttcacccgtggctgccgtcactcctcccctgttcgctgcatctggactgcatgctaatgccaatcttagtgctgaccagtctccatctgcactgagcgctggtaaaactgcagttaatgtcccacggcagcccacagtcaccagcgtgtgttccgagacttctcaggagctagcagagggacagagaagaggatcccagggtgacagtaaacagcaaaatggcccaccaagaaaacacattgtggagcgctatacagagttttatcatgtacccactcacagtgatgccagcaagaagagactgattgaggatactgaagactggcgtccaaggactggaacaactcagtctcgctctttccgaatccttgcccagatcactgggactgaacatttgaaagaatctgaagccgataatacaaagaaggcaaataactctcaggagccttctccgcagttggcttcctcggtagcttccacacggagcatgcccgagagcctggacagcccaacctctggcagaccaggggttaccagcctcacaactgcagctgccttcaagcctgtaggatccactggcgtcatcaagtcaccaagctggcaacggccaaaccaaggagtaccttccactggaagaatctcaaacagcgctacttactcaggatcagtggcaccagccaactcagctttgggacaaacccagccaagtgaccaggacactttagtgcaaagagctgagcacattccagcagggaaacgaactccgatgtgcgcccattgtaaccaggtcatcagaggaccattcttagtggcactggggaaatcttggcacccagaagaattcaactgcgctcactgcaaaaatacaatggcctacattggatttgtagaggagaaaggagccctgtattgtgagctgtgctatgagaaattctttgcccctgaatgtggtcgatgccaaaggaagatccttggagaagtcatcaatgcgttgaaacaaacttggcatgtttcctgttttgtgtgtgtagcctgtggaaagcccattcggaacaatgtttttcacttggaggatggtgaaccctactgtgagactgattattatgccctctttggtactatatgccatggatgtgaatttcccatagaagctggtgacatgttcctggaagctctgggctacacctggcatgacacttgctttgtatgctcagtgtgttgtgaaagtttggaaggtcagacctttttctccaagaaggacaagcccctgtgtaagaaacatgctcattctgtgaatttttga

SEQ ID NO: 4 - protein, 596 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKGEPKEVVKPVPITSPAVSKVTSTNNMAYNKAPRPFGSVSSPKVTSIPSPSSAFTPAHATTSSHASPSPVAAVTPPLFAASGLHANANLSADQSPSALSAGKTAVNVPRQPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 5 - DNA, 1464 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagcccacagtcaccagcgtgtgttccgagacttctcaggagctagcagagggacagagaagaggatcccagggtgacagtaaacagcaaaatggcccaccaagaaaacacattgtggagcgctatacagagttttatcatgtacccactcacagtgatgccagcaagaagagactgattgaggatactgaagactggcgtccaaggactggaacaactcagtctcgctctttccgaatccttgcccagatcactgggactgaacatttgaaagaatctgaagccgataatacaaagaaggcaaataactctcaggagccttctccgcagttggcttcctcggtagcttccacacggagcatgcccgagagcctggacagcccaacctctggcagaccaggggttaccagcctcacaactgcagctgccttcaagcctgtaggatccactggcgtcatcaagtcaccaagctggcaacggccaaaccaaggagtaccttccactggaagaatctcaaacagcgctacttactcaggatcagtggcaccagccaactcagctttgggacaaacccagccaagtgaccaggacactttagtgcaaagagctgagcacattccagcagggaaacgaactccgatgtgcgcccattgtaaccaggtcatcagaggaccattcttagtggcactggggaaatcttggcacccagaagaattcaactgcgctcactgcaaaaatacaatggcctacattggatttgtagaggagaaaggagccctgtattgtgagctgtgctatgagaaattctttgcccctgaatgtggtcgatgccaaaggaagatccttggagaagtcatcaatgcgttgaaacaaacttggcatgtttcctgttttgtgtgtgtagcctgtggaaagcccattcggaacaatgtttttcacttggaggatggtgaaccctactgtgagactgattattatgccctctttggtactatatgccatggatgtgaatttcccatagaagctggtgacatgttcctggaagctctgggctacacctggcatgacacttgctttgtatgctcagtgtgttgtgaaagtttggaaggtcagacctttttctccaagaaggacaagcccctgtgtaagaaacatgctcattctgtgaatttttga

SEQ ID NO: 6 - protein, 487 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTTAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVINALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 7 - DNA, 354 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagcccaccaagaaaacacattgtggagcgctatacagagttttatcatgtacccactcacagtga

SEQ ID NO: 8 - protein, 117 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKPTKKTHCGALYRVLSCTHSQ

SEQ ID NO: 9 - DNA, 705 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagcccacagtcaccagcgtgtgttccgagacttctcaggagctagcagagggacagagaagaggatcccagggtgacagtaaacagcaaaatgggaaaatcccacctaaacgcccaccaagaaaacacattgtggagcgctatacagagttttatcatgtacccactcacagtgatgccagcaagaagagactgattgaggatactgaagactggcgtccaaggactggaacaactcagtctcgctctttccgaatccttgcccagatcactgggactgaacatttgaaagaatctgaagccgataatacaaagaaggcaaaggaaaagataccccttcacgtctttagtcccaaatacacaaaattacgtgactggcaccatgaagtttcagcacgtgctcttaacgtacagtga

SEQ ID NO: 10 - protein, 234 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKPTVTSVCSETSQELAEGQRRGSQGDSKQQNGKIPPKRPPRKHIVERYTEFYHVPTHSDASKKRLIEDTEDWRPRTGTTQSRSFRILAQITGTEHLKESEADNTKKAKEKIPLHVFSPKYTKLRDWHHEVSARALNVQ

SEQ ID NO: 11 - DNA, 645 bp, Homo sapiens
atgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagaaaacacaagtgacaaataaccctggcactgtgaaaatcccacctaaacgcccaccaagaaaacacattgtggagcgctatacagagttttatcatgtacccactcacagtgatgccagcaagaagagactgattgaggatactgaagactggcgtccaaggactggaacaactcagtctcgctctttccgaatccttgcccagatcactgggactgaacatttgaaagaatctgaagccgataatacaaagaaggcaaaggaaaagataccccttcacgtctttagtcccaaatacacaaaattacgtgactggcaccatgaagtttcagcacgtgctcttaacgtacagtga

SEQ ID NO: 12 - protein, 214 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKKTQVTNNPGTVKIPPKRPPRKHIVERYTEFYHVPTHSDASKKRLIEDTEDWRPRTGTTQSRSFRILAQITGTEHLKESEADNTKKAKEKIPLHVFSPKYTKLRDWHHEVSARALNVQ